(19) United States
(12) Patent Application Publication
(10) Pub. No.: US 2026/0216213 A1
(43) Pub. Date: Jul. 30, 2026

(54) METHODS FOR TREATING ROSACEA

(71) Applicant: JOURNEY MEDICAL CORPORATION, Scottsdale, AZ (US)

(72) Inventors: Srinivas Ramchandra SIDGIDDI, West Windsor, NJ (US); Anirudh GAUTAM, Aesch BL (CH)

(21) Appl. No.: 19/374,820

(22) Filed: Oct. 30, 2025

Related U.S. Application Data
(60) Provisional application No. 63/715,230, filed on Nov. 1, 2024.

(51) Int. Cl. A61K 31/65; A61P 17/00

(52) U.S. Cl. CPC A61K 31/65; A61P 17/00

(57) ABSTRACT

A pharmaceutical composition for treating moderate or severe rosacea comprises a formulated, body weight-independent low dose of minocycline that reduces the severity of rosacea in at least 2 weeks with a decrease in lesion count, Investigator's Global Assessment (IGA) score, and Clinician's Erythema Assessment (CEA) score with a measured improvement in quality of life.

### CROSS-REFERENCE TO RELATED APPLICATION(S)

This application claims priority to and the benefit of U.S. Provisional Application No. 63/715,230, filed Nov. 1, 2024, the entire content of which is herein incorporated by reference.

### FIELD

The present application relates to a method of treating rosacea by administering a pharmaceutical composition comprising a body weight-independent dose of a formulated minocycline composition to a subject in need thereof, wherein said administration provides an effective dermal mean concentration of minocycline for effectively treating moderate to severe rosacea, while maintaining low plasma concentrations.

### BACKGROUND

Rosacea is a chronic inflammatory disorder characterized by facial flushing, telangiectasias, erythema, papules, pustules, and in severe cases, rhinophyma. Rosacea can be associated with the elevated levels of cathelicidins or with the elevated levels of a stratum corneum tryptic enzyme (SCTE). Rosacea includes any of the known types or subtypes classified in the art.

Overall, rosacea is a type of inflammation that shows up as a rash, similar to other rashes/breakouts that cause an area of red, sensitive and inflamed skin. Such rashes are caused by irritation, allergies, infections, underlying diseases and structural defects of the skin, including blocked pores or malfunctioning oil glands. With time, people who have rosacea often see permanent redness in the center of their face.

There are at least four different types of rosacea, although some people will have symptoms from more than one type at a time. The four types of rosacea are:
1. Erythematotelangiectatic rosacea: Redness, flushing, visible blood vessels,
2. Papulopustular rosacea: Redness, swelling, and acne-like breakouts,
3. Phymatous rosacea: Skin thickens and has a bumpy texture, redness and various symptoms from other subtypes, and
4. Ocular rosacea: Eyes red and irritated, eyelids can be swollen, and person may have what looks like a stye.

There are several subtypes of rosacea including, for example, but not limited to, pyoderma faciale (also known as rosacea fulminans), rosacea conglobate or phymatous rosacea.

Thus, a rosacea related disorder is any disorder which can occur in parallel with rosacea or be a contributing factor to the outbreak of rosacea or can resemble rosacea. Perioral dermatitis is an erythematous, papulopustular facial eruption that resembles rosacea and/or acne but typically starts around the nose.

There are various treatment options available for inflammatory skin conditions like rosacea. It is treated with a variety of topical and/or systemic therapies including topical and oral antibiotics, ivermectin, sodium sulfacetamide, and metronidazole.

Topical azelaic acid is an alternative to topical antibiotics to treat mild-to-moderate spots. However, some people find that it can cause side-effects such as burning, stinging, itching, scaling and dry skin.

When antibiotics are ineffective or poorly tolerated, oral isotretinoin may be effective; however, it has various side effects which are not suitable for everyone. Ivermectin cream is used occasionally for people with rosacea. It works by killing the mite Demodex folliculorum and also works to reduce some of the inflammation in the skin. Brimonidine gel is used to treat facial redness. It results in short-term vasoconstriction but has no effects on telangiectasia. Certain medications such as clonidine (an alpha2-receptor agonist) may reduce the vascular dilatation (widening of blood vessels) that results in flushing. Oral non-steroidal anti-inflammatory agents such as diclofenac may reduce the discomfort and redness of affected skin. Calcineurin inhibitors such as tacrolimus ointment and pimecrolimus cream are reported to help some subjects with rosacea. Other than medications, persistent telangiectasia has shown improvements with surgical treatments like vascular laser or intense pulsed light treatment. Papulopustular rosacea may also improve with laser treatment or radiofrequency. Other treatments like cautery, diathermy (electrosurgery) or sclerotherapy (strong saline injections) may also be helpful. Nutraceuticals targeting flushing, facial redness and inflammation may be beneficial.

Among various available treatment options, oral antibiotics like doxycycline is preferred, which reduces inflammation significantly. The oral antibiotics may reduce the redness, papules, pustules and eye symptoms of rosacea. The antibiotics are usually prescribed for 6 to 16 weeks, with the duration depending on the severity of the rosacea. Further courses are often needed from time to time, as the antibiotics do not cure the disorder. Sometimes other oral antibiotics such as cotrimoxazole or metronidazole are prescribed in cases of resistance to doxycycline. Anti-inflammatory effects of antibiotics are under investigation and shown to inhibit matrix. metalloproteinases function and in turn reduce cathelicidins and inflammation.

Currently in the United States of America, oral doxycycline is marketed under the brand name ORACEA®—40 mg once daily capsules—from Galderma Laboratories, approved by the US FDA in 2006. ORACEAR is indicated for the treatment of only inflammatory lesions (papules and pustules) of rosacea in adults. However, ORACEA® has only modest efficacy and well-known esophageal irritation and ulceration related side effects that are not tolerated by or acceptable to many rosacea subjects.

The usage of minocycline in higher strengths (i.e., 1 mg/kg or higher) is associated with vestibular deregulation related adverse events like vertigo, incoordination or light-headedness. There is a clear void in the area of treating inflammatory skin conditions like rosacea in terms of safe and commercially approved minocycline dosage forms. Currently, there is no approved dosage form of minocycline available for treating inflammatory skin conditions like rosacea.

There is a clear unmet need to provide alternatives for treatment of inflammatory skin conditions, which increase the opportunities for a greater number of individuals to achieve effective treatment. In this regard, there is a need for methods of providing an effective treatment of rosacea that maximizes therapeutic effect in an efficient time period, while managing the adverse effects.

In other words, there is a need to provide minocycline dosage forms for better compliance to effectively and efficiently treat rosacea, which minimize the fluctuation or variance between peak and trough plasma or interstitial fluid levels of minocycline, with reduced side effects.

It is well known in the art that peak-to-trough fluctuations of drug concentration critically affect clinical response, tolerability and ultimately selection of appropriate drug and dosage forms required for treatment of inflammatory skin conditions.

### SUMMARY

The present application provides a pharmaceutical composition comprising a body weight-independent reduced dose formulation of minocycline for treating rosacea, and a method of treating rosacea by administering the pharmaceutical composition on a daily basis for at least 2 weeks, to a subject suffering from rosacea to provide an effective treatment of the rosacea.

Accordingly, the present application relates to a method of treating or a pharmaceutical composition for use in treating moderate to severe rosacea by administering a pharmaceutical composition comprising a reduced dose of minocycline comprising a therapeutically effective amount of minocycline in a formulation capable of reducing the severity of rosacea after at least 2 weeks of daily administration to a subject in need thereof, wherein the effective amount of the minocycline is independent of the subject's weight and the treatment includes daily, oral administration of about 20 to 40 mg of the minocycline composition as disclosed herein, and wherein moderate to severe rosacea is characterized by an Investigator's Global Assessment (IGA) score of 3 to 4, respectively.

In some embodiments, the pharmaceutical composition reduces the IGA score by at least 2 grades. In some embodiments, the pharmaceutical composition reduces the IGA score to 0 or 1.

In some embodiments, the pharmaceutical composition comprises about 30 mg to about 40 mg minocycline. In other embodiments, the pharmaceutical composition comprises about 40 mg minocycline.

In some embodiments, the pharmaceutical composition for use in treating moderate to severe rosacea, comprises an immediate release (IR) portion and an extended release (ER) portion with an IR to ER ratio ranging from 1:1 to 1:4. In other embodiments, the pharmaceutical composition comprises an immediate release (IR) portion and an extended release (ER) portion with an IR to ER ratio of 1:3 or 25:75.

In some embodiments, the method or the use comprises orally administration on a daily basis for 2 weeks, 4 weeks, 8 weeks, 12 weeks, or 16 weeks.

In some embodiments, the method of treating or a pharmaceutical composition for use in treating moderate to severe rosacea in a subject comprises a subject having Fitzpatrick skin type of I, II, III, IV, V, or VI.

In some embodiments, a method of treating or a pharmaceutical composition for the use in treating moderate to severe rosacea in a subject suffering therefrom, includes orally administering the pharmaceutical composition comprising about 20 mg to about 40 mg of minocycline to the subject on a daily basis, wherein upon orally administering the pharmaceutical composition for at least 2 weeks and up to 16 weeks, the severity of the rosacea is reduced with a decrease in lesion count as compared to the lesion count before administering the pharmaceutical composition.

In some embodiments, the severity of the rosacea is reduced with a decrease in lesion count by at least 20% in 2 weeks after 2 weeks of daily oral administration of a 40 mg minocycline composition as disclosed herein. In other embodiments, the severity of the rosacea is reduced with a decrease in lesion count by at least 40% in 2 weeks. In still other embodiments, the severity of the rosacea is reduced with a decrease in lesion count by at least 65% in 16 weeks.

In some embodiments, the subject has an Investigator's Global Assessment (IGA) score of 3 (moderate) or 4 (severe), and the reduction in the severity of the rosacea comprises a reduction in IGA by at least one grade after 2 weeks of daily oral administration of a 40 mg minocycline composition as disclosed herein. In other embodiments, the IGA is reduced by at least two grades at 2 weeks. In further embodiments, the IGA score is 0 or 1 at 2 weeks, 4 weeks, 8 weeks, 12 weeks, or 16 weeks.

In some embodiments, the severity of the rosacea is reduced with the administration of a 40 mg minocycline composition as disclosed herein, wherein the severity of the rosacea is reduced with a decrease in lesion count, and wherein the severity of the rosacea before treatment comprises at least 15 facial rosacea lesions.

In some embodiments, a method of treating or a pharmaceutical composition for the use in treating moderate to severe rosacea in a subject suffering therefrom, includes orally administering the pharmaceutical composition comprising about 20 mg to about 40 mg of minocycline to the subject on a daily basis, wherein upon orally administering the pharmaceutical composition for at least 2 weeks, the reduction in the severity of the rosacea comprises a reduction in Clinicians Erythema Assessment (CEA) score. In some embodiments, the CEA score is reduced by at least 2 grades. In other embodiments, the CEA score is reduced by at least 2 grades in 2, 4, 8, 12, or 16 weeks,

In some embodiments, a method of treating or a pharmaceutical composition for the use in treating moderate to severe rosacea in a subject suffering therefrom, includes orally administering the pharmaceutical composition comprising about 40 mg of minocycline to the subject on a daily basis, wherein upon orally administering the pharmaceutical composition for at least 2 weeks and up to 16 weeks, results in an improvement in quality of life as indicated by a decrease in a Rosacea Quality of Life score and/or a decrease in a Dermatology Life Quality Index score.

In some embodiments, a method of treating or a pharmaceutical composition for the use in treating moderate to severe rosacea in a subject suffering therefrom, includes orally administering the pharmaceutical composition comprising about 20 mg to about 40 mg of minocycline to the subject on a daily basis for 2 to 16 weeks, wherein the plasma and dermal mean concentrations of the minocycline in the subjects do not increase by more than 20% relative to Day 1.

In another aspect of the above embodiments, the present application relates to a method of treating or use of an oral pharmaceutical composition to treat moderate to severe rosacea by administering the oral pharmaceutical composition comprising a body weight-independent dose of about 40 mg of minocycline in a formulation having an immediate release (IR) portion and an extended release (ER) portion with an IR to ER ratio ranging from 1:1 to 1:4, the administration to a subject on a daily basis in need thereof for at least 2 weeks and up to 16 weeks, wherein said administration for at least 2 weeks results in a decrease in severity of the rosacea as disclosed herein. In other embodiments, the pharmaceutical composition comprises an immediate release (IR) portion and an extended release (ER) portion in an IR to ER ratio of 1:3 or 25:75. In still other embodiments, the pharmaceutical composition having an IR portion and an ER portion comprises minocycline hydrochloride layered onto inert cores.

In an embodiment, the present application relates to a method of treating or a pharmaceutical composition for the use in treating moderate or severe rosacea by administering to a subject suffering from rosacea the oral pharmaceutical composition as disclosed herein, comprising a body weight-independent dose of about 40 mg of minocycline in the form of a composition having an IR portion and an ER portion, the oral pharmaceutical composition administered on a daily basis for 2, 4, 8, 12, or 16 weeks, as assessed using Investigator's Global Assessment (IGA) scale, wherein moderate rosacea has an IGA grade of 3 and severe rosacea has an IGA grade of 4.

In an embodiment, the present application relates to a method of treating or a pharmaceutical composition for the use in treating moderate or severe rosacea by administering to a subject suffering from rosacea an oral pharmaceutical composition as disclosed herein, comprising a body weight-independent dose of about 40 mg of minocycline on a daily basis for 2, 4, 8, 12, or 16 weeks, resulting in a reduction in the severity of rosacea by at least two IGA grades. Treatment of the daily administration of the disclosed minocycline composition may continue beyond 2 weeks for 4, 8, 12, or 16 weeks.

In an alternative or additional embodiment, said method of administering the oral pharmaceutical composition or the use of the oral pharmaceutical composition comprises a reduced dose of about 40 mg of minocycline that results in an equivalent or more effective treatment of rosacea in a subject compared to administering 40 mg doxycycline to the subject suffering from rosacea.

In another aspect of the above embodiments, the pharmaceutical composition of present application is a formulation of minocycline as disclosed herein and prepared in the form of oral tablets, capsules, pills, minitablets, pellets, granules, powder, suspension or syrup.

In an embodiment, the present application relates to a method of treating rosacea by administering an oral pharmaceutical composition comprising a reduced dose of minocycline to a subject in need thereof, wherein said method provides body weight-independent dosing regimen for minocycline . . .

In an embodiment, the present application relates to a pharmaceutical composition comprising 40 mg of minocycline formulated as disclosed herein, wherein after 3 weeks (21 days) of oral administration of the pharmaceutical composition to a subject in need thereof, the interstitial fluid (ISF) AUC₀₋₂₄ and Cₘₐₓ in the subject on Day 21 do not increase by more than 20% compared to Day 1.

In an embodiment, the present application relates to a pharmaceutical composition comprising 40 mg of minocycline formulated as disclosed herein, wherein after 3 weeks (21 days) of oral administration of the pharmaceutical composition to a subject in need thereof, the interstitial fluid (ISF) AUC₀₋₂₄ and Cₘₐₓ in the subject at Day 1 is at least 80% of the ISF AUC₀₋₂₄ and Cₘₐₓ in the subject at Day 21.

In an embodiment, the present application relates to a pharmaceutical composition comprising 40 mg of minocycline formulated as disclosed herein, wherein after 3 weeks (21 days) of oral administration of the pharmaceutical composition to a subject in need thereof, the plasma AUC₀₋₂₄ and Cₘₐₓ in the subject at Day 1 and Day 21 differ by 15% or less.

In an embodiment, the present application relates to a pharmaceutical composition comprising 20 mg of minocycline formulated as disclosed herein, wherein after 3 weeks (21 days) of oral administration of the pharmaceutical composition to a subject in need thereof, the interstitial fluid (ISF) Cₘₐₓ in the subject increases by about 41% and the plasma Cₘₐₓ increases by about 25% on Day 21 compared to Day 1.

### BRIEF DESCRIPTION OF THE DRAWINGS

FIGS. 1A and 1B are line graphs showing the mean plasma concentration in subjects after daily, oral administration of 20 mg minocycline (Mino 20 mg, triangles), 40 mg minocycline (Mino 40 mg, squares), or 40 mg doxycycline (Doxy 40 mg, circles) at 2 hour intervals for 24 hours on Day 1 (FIG. 1A) and Day 21 (FIG. 1B), as set forth in Example 2, according to embodiments of the present invention.

FIGS. 2A and 2B are line graphs showing the mean interstitial fluid (ISF) (dermal) concentration in subjects after daily, oral administration of 20 mg minocycline (Mino 20 mg, triangles), 40 mg minocycline (Mino 40 mg, squares), or 40 mg doxycycline (Doxy 40 mg, circles) at 2 hour intervals for 24 hours on Day 1 (FIG. 2A) and Day 21 (FIG. 2B), as set forth in Example 2, according to embodiments of the present invention.

FIGS. 3A and 3B are bar graphs from the MVOR-1 and MVOR-2 studies, respectively, showing “treatment success” (the percent of subjects with an IGA grade of 0 or 1 and at least a 2-grade IGA reduction) after daily, oral administration of DFD-29 (minocycline at 40 mg, dark shading), Oracea (doxycycline at 40 mg, hatched lines), or placebo (light shading) for 2 weeks, 4, weeks, 8 weeks, 12 weeks, and 16 weeks, as indicated, as set forth in Example 4, according to embodiments of the present invention.

FIGS. 4A and 4B are bar graphs showing reduction in total inflammatory lesion count after daily, oral administration of DFD-29 (minocycline at 40 mg, dark shading), Oracea (doxycycline at 40 mg, hatched lines), or placebo (light shading) for 2 weeks, 4, weeks, 8 weeks, 12 weeks, and 16 weeks, as indicated, as set forth in Example 5, according to embodiments of the present invention.

FIGS. 5A and 5B are bar graphs showing the percent of subjects with at least a 2-grade reduction in Clinician's Erythema Assessment (CEA) score from Baseline to Week 16, as set forth in Example 6, according to embodiments of the present invention.

### DETAILED DESCRIPTION

Aspects of embodiments of the present invention are directed to a pharmaceutical composition for the use in treating or a method of treating moderate to severe rosacea in a subject suffering therefrom, in which the pharmaceutical composition comprising a formulation of about 20 mg to about 40 mg of minocycline is orally administered for at least 2 weeks to the subject, thereby decreasing the severity of the rosacea in the subject.

The details of one or more embodiments of the present invention are set forth in this document.

Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control,

### Definitions

The terms as used herein have the following meanings:

The present invention can comprise or consist essentially of the components of the present invention as well as other ingredients or elements described herein. As used herein, “comprising” means the elements recited, or their equivalent in structure or function, plus any other element or elements which are not recited. The terms “having,” “including,” and “comprised of” are also to be construed as open ended unless the context suggests otherwise. As used herein. “consisting essentially of” means that the invention may include ingredients in addition to those recited in the claim, but only if the additional ingredients do not materially alter the basic and novel characteristics of the claimed invention. As used herein, “consisting of” means that the invention excludes additional elements, steps, or ingredients not specified in the claim.

The terms “a” and “the” as used herein, are understood to encompass the plural as well as the singular or otherwise clearly mentioned wherever needed. For example, reference to “an excipient” includes reference to one or more of such excipients, and reference to “the vehicle” includes reference to one or more of such vehicles.

The terms “about,” “up to,” “generally,” and the like are to be construed as modifying a term or value such that it is not an absolute. Such terms will be defined by the circumstances and the terms that they modify as those terms are understood by those skilled in the art. This includes, at very least, the degree of expected experimental error, technical error and instrumental error for a given experiment, technique or an instrument used to measure a value.

The term “about” is used to provide flexibility to a numerical range endpoint by providing that a given value may be “a little above” or “a little below” the endpoint. For example, “about” includes up to 5.0% more or less of the value it precedes, such that “about 40 mg” includes from 5% less than 40 mg up to 5% more than 40 mg. Accordingly, “about” includes up to 0.1, 0.5%, 1.0%, 1.5%, 2.0%, 2.5%, 3.0%, 3.5%, 4.0%, 4.5%, and 5.0% more or less than the value it precedes. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum.

The terms “composition” and “formulation” refer to a mixture of two or more compounds, elements, or molecules. Also, this term may be used to refer to a mixture of one or more active agents with a pharmaceutically acceptable vehicle or excipients. Furthermore, the term “dosage form” can include one or more formulation(s) or composition(s) provided in a format for oral administration like tablets, capsules, pills, minitablets, pellets, granules, powder, suspension, syrup and the like or mixtures thereof.

As used herein, the terms “optional” or “optionally” mean that the subsequently described event or circumstance does or does not occur or exist and that the description includes instances where said event or circumstance occurs or exists, and instances where it does not.

As used herein, the terms “treatment” or “treating” that includes “alleviating,” relate to curing or substantially curing a condition, as well as ameliorating at least one symptom of the condition, and are inclusive of prophylactic treatment and therapeutic treatment. As would be recognized by one or ordinary skill in the art, treatment that is administered prior to clinical manifestation of a condition then the treatment is prophylactic (i.e., it protects the subject against developing the condition). If the treatment is administered after manifestation of the condition, the treatment is therapeutic (i.e., it is intended to diminish, ameliorate, control, alleviate or maintain the existing condition and/or side effects associated with the condition). The terms relate to medical management of a subject with the intent to substantially cure, ameliorate, stabilize, or substantially prevent a condition, including but not limited to prophylactic treatment to preclude, avert, obviate, forestall, stop, or hinder something from happening, or reduce the severity of something happening, especially by advance action. As such, the terms treatment or treating include, but are not limited to: inhibiting the progression of a condition of interest; arresting or preventing the development of a condition of interest; reducing the severity of a condition of interest; ameliorating or relieving symptoms associated with a condition of interest; causing a regression of the condition of interest or one or more of the symptoms associated with the condition of interest; and preventing a condition of interest or the development of a condition of interest.

The term “minocycline” as used herein, is intended to include, but is not limited to, minocycline, its pharmaceutically acceptable salts, and pharmaceutically acceptable, pharmacologically active derivatives of minocycline, including both individual enantiomers of minocycline (dextrogyral and levogyral enantiomers) in their substantially pure form and their pharmaceutically acceptable salts, mixtures (in any ratio) of minocycline enantiomers and their pharmaceutically acceptable salts, and active metabolites of minocycline and their pharmaceutically acceptable salts. The chemical name of minocycline is [4S-(4α,4aα,5aα, 12aα)]-,7-Bis(dimethylamino)-1,4-,4a,5,5a,6,11,12a-octahydro-3,10,12,12a-tetrahydroxy-1,11-dioxo-2-naphthac-enecarboxamide. The solid state form of minocycline can be used. Alternatively, for example, minocycline can be amorphous or crystalline.

The term “doxycycline” as used herein, is intended to include, but is not limited to, doxycycline, its pharmaceutically acceptable salts, and pharmaceutically acceptable, pharmacologically active derivatives of doxycycline, including both individual enantiomers of doxycycline in their substantially pure form and their pharmaceutically acceptable salts, mixtures (in any ratio) of doxycycline enantiomers and their pharmaceutically acceptable salts, and active metabolites of doxycycline and their pharmaceutically acceptable salts. The chemical name of doxycycline is (4S,4aR,5S,5aR,6R,12aR)-4-(dimethylamino)-1,5,10,11,12a-pentahydroxy-6-me-thyl-3,12-dioxo-4a,5,5a,6-tetrahydro-4H-tetracene-2-carboxamide. The solid state form of doxycycline can be used. Alternatively, for example, doxycycline can be amorphous or crystalline.

The term “doxycycline composition” as used herein, includes an oral doxycycline marketed under the brand name as ORACEA®—40 mg once daily capsules—from Galderma Laboratories, approved by the US FDA with NDA No. 050805 and indicated for the treatment of only inflammatory lesions (papules and pustules) of rosacea in adults. The doxycycline composition includes ORACEA® or its pharmaceutical equivalents or its therapeutic equivalents or later approved drugs which are designated as AB rated by US FDA as per Approved Drug Products with Therapeutic Equivalence Evaluations (34th edition) or drugs obtained marketing approval by US FDA through Abbreviated New Drug Application (ANDA) filing by establishing bioequivalence.

The term “commercially available minocycline compositions” as used herein, includes oral minocycline marketed under the brand name as SOLODYN®−1 mg/kg once daily (45 mg, 55 mg, 65 mg, 80 mg, 90 mg, 105 mg, 115 mg, and 135 mg tablets)—from Medicis Pharmaceutical Corp., approved by the US FDA with NDA No. 050808 and indicated to treat only inflammatory lesions of non-nodular moderate to severe acne vulgaris in patients 12 years of age and older. The commercially available minocycline compositions includes SOLODYN® or its pharmaceutical equivalents or its therapeutic equivalents or later approved drugs which are designated as AB rated by US FDA as per Approved Drug Products with Therapeutic Equivalence Evaluations (34th edition) or drugs obtained marketing approval by US FDA through Abbreviated New Drug Application (ANDA) filing by establishing bioequivalence.

The term “pharmaceutically acceptable salts” as used herein, includes those salts which are, within the scope of sound medical judgment, suitable for use in contact with the tissues of humans and lower animals without undue toxicity, irritation, allergic response and the like, which are well known in the art. The salts can be prepared in situ during the final isolation and purification of the compounds of the invention, or separately by reacting the pharmaceutically active substance having a free base function with a suitable organic acid or inorganic acid. Examples of pharmaceutically acceptable salts include, but are not limited to, any of the salts or co-crystals of minocycline selected from hydrochloride, hydrobromide, sulphate, citrate, phosphate, maleate, formate, acetate, nitrate, mesylate, succinate, benzoate, and the like. The salts may be in solvate, hydrate, hemihydrates, or anhydrous forms.

The term “therapeutically effective amount” as used herein, refers to a prescribed amount of minocycline, which is less than 45 mg. In some embodiments, the therapeutically effective amount of minocycline is about 44 mg, about 43 mg, about 42 mg, about 41 mg, about 40 mg, about 39 mg, about 38 mg, about 37 mg, about 36 mg, about 35 mg, about 34 mg, about 33 mg, about 32 mg, about 31 mg, about 30 mg, about 29 mg, about 28 mg, about 27 mg, about 26 mg, about 25 mg, about 24 mg, about 23 mg, about 22 mg, about 21 mg, about 20 mg, about 19 mg, about 18 mg, about 17 mg, about 16 mg, about 15 mg, about 14 mg, about 13 mg, about 12 mg, about 11 mg, or about 10 mg. Most typically, the therapeutically effective amount of minocycline is 40 mg. Such therapeutically effective amount provides a sufficient plasma and/or mean interstitial fluid (dermal) concentration of minocycline for treating rosacea. The specified amount of minocycline is expected to be low enough to avoid serious side effects.

The term “reduced dose” refers to a dose of minocycline, wherein said dose comprises less than 45 mg of minocycline. In some embodiments, said reduced dose of minocycline comprises about 40 mg, about 35 mg, about 30 mg, about 25 mg, about 20 mg, about 15 mg or about 10 mg of minocycline. In some embodiments, the reduced dose of minocycline is about 44 mg, about 43 mg, about 42 mg, about 41 mg, about 40 mg, about 39 mg, about 38 mg, about 37 mg, about 36 mg, about 35 mg, about 34 mg, about 33 mg, about 32 mg, about 31 mg, about 30 mg, about 29 mg, about 28 mg, about 27 mg, about 26 mg, about 25 mg, about 24 mg, about 23 mg, about 22 mg, about 21 mg, about 20 mg, about 19 mg, about 18 mg, about 17 mg, about 16 mg, about 15 mg, about 14 mg, about 13 mg, about 12 mg, about 11 mg, or about 10 mg of minocycline. Most typically, the reduced dose of minocycline is 40 mg. The term “reduced dose” can also be used herein to refer comparisons between minocycline and doxycycline. In this regard, minocycline that is provided at a reduced dose as compared to doxycycline at a reduced mg amount. For example, less than 40 mg of minocycline is a reduced dose as compared to 40 mg doxycycline. Similarly, the term “equivalent dose” can be used to refer to comparisons between minocycline and doxycycline. In this regard, minocycline that is provided at an equivalent dose as compared to doxycycline is of the same mg amount. For example, 40 mg of minocycline is an equivalent dose to 40 mg doxycycline.

The term “sub-antimicrobial dose” refers to an amount having no significant antimicrobial effect on the body upon administration of such amount.

The term “rosacea” as used herein, includes a skin condition comprising inflammatory lesions (papulopustular rosacea--papules, pustules and nodules), vascular instability and/or vascular ectasia (erythematotelangiectatic rosacea), edema, skin thickening and/or rhinophyma changes (phymatous rosacea), acne rosacea, or ocular changes (ocular rosacea), wherein the full scope of rosacea, including its causes, symptoms, and effects as described by National Rosacea Society, which is a 501 (c) (3) organization in the United States.

The term “subject” as used herein, refers to a human individual who may or may not be suffering from an inflammatory skin condition, who is a recipient of an oral pharmaceutical composition described herein.

The term “patient” as used herein, refers to a human individual who is suffering from an inflammatory skin condition, who is a recipient of the oral pharmaceutical composition described herein.

The term “efficacy” refers to a reduction or inhibition of severity of rosacea, wherein said severity is assessed by counting inflammatory lesions, including papules, pustules and/or nodules, before the onset of treatment; and counting inflammatory lesions after treatment has been initiated; and assessing changes in an investigator's global assessment (IGA) score and/or in the number of inflammatory lesions. If desired, efficacy can be quantified by reduction of inflammatory lesion count from the baseline before treatment, by reduction from the baseline in an IGA score, or by both the reduction of inflammatory lesion count and the IGA score. As used herein, the term “treatment success” refers to an IGA score of 0 or 1 and at least a 2-grade reduction in IGA score.

The term “equivalent efficacy” refers to comparative treatment using two different compositions, wherein the resulting efficacy is substantially the same. For example, if treatment with two different compositions for the same period of time results in substantially the same reduction in the number of lesions, the compositions can be said to have equivalent or comparable efficacy.

The term “improved efficacy” refers to comparative treatment using two different compositions, wherein the resulting efficacy of one composition is greater as compared to another composition. For example, if treatment with a first composition and a second composition for the same period of time results in greater reduction in the number of inflammatory lesions for the second composition as compared to the first composition, the second composition is said to have improved efficacy as compared to the first composition.

The term “substantially” can be used to modify a term or value such that it is not an absolute. The term will be defined by the circumstances and the terms that it modifies, as those terms are understood by those skilled in the art. The term is indicative of an approximation or some amount of deviation, rather than perfect and absolute. For example, as will be recognized by those skilled in the art, when the term “substantially prevent” is used in connection with a prophylactic treatment, it should not be understood as an absolute term that would preclude any sign of any skin condition in a subject. Rather, as used in the context of prophylactic treatment, the term “substantially prevent” can refer to inhibiting the development of a skin condition, such as in a subject who may be predisposed to the skin condition but who has not yet been diagnosed as having it, limiting the severity of the developed skin condition, arresting the development of a skin condition, and the like.

The term “investigator's global assessment (IGA) score” as used herein is determined by a trained medical professional evaluating the skin condition of a subject utilizing an investigative global assessment of the skin condition. Typically, such global assessments assign a value to the degree of rosacea exhibited by the skin. For example, the IGA score for rosacea (Table 1) can range from 0 (clear) to 1 (almost clear) to 2 (mild) to 3 (moderate) to 4 (severe),

TABLE 1

| Score | Definition |
|---|---|
| 0 = Clear | No signs or symptoms present |
| 1 = Near clear | Rare papules |
| 2 = Mild | Some papules and pustules with no plaques |
| 3 = Moderate | Several small/large papules and pustules, |
| | with no plaques |
| 4 = Severe | Numerous small/large papules and pustules, |
| | with or without plaques and nodules |

The reduction in inflammatory lesion count is determined at each study visit inter alia by an absolute inflammatory lesion count or by a percentage change in inflammatory lesion count. The improvement assessment includes grading or scoring rosacea severity based on the total count or number of inflammatory lesions.

The term “moderate to severe rosacea” refers to at least about 15 papulopustular lesions before treatment. For example, the subject can have an IGA score of rosacea of about 3 or about 4, and at least about 15, 20, 25 or more inflammatory papulopustular lesions before treatment. As used herein, a subject having “moderate rosacea” has an IGA score of rosacea of about 3. As used herein, a subject having “severe rosacea” has an IGA score of rosacea of about 4.

The term “interstitial fluid” or “ISF” as used herein refers to the extracellular fluid that is located outside blood vessels and in spaces between the tissue cells, and does not contain blood and blood cells, but may contain non-cellular blood components. The “ISF concentration” is used herein to refer to the “dermal concentration” in contrast to the “plasma concentration.”

The term “plasma concentration” as used herein refers to the concentration of minocycline or doxycycline found in blood; and may also be referred to as “systemic exposure.” The plasma concentration is disclosed in contrast to the ISF or dermal concentration.

The term “steady state” as used herein refers to a concentration level of minocycline in which there is no further difference, or having minimal difference, between the peak and trough concentrations of minocycline, in plasma or interstitial fluid. Thus, at steady state, the plasma or interstitial fluid concentration level of minocycline does not substantially fluctuate within the dosing interval after repeated doses of the formulation. The steady state can also be referred to as “equilibration,” and is achieved in about 3 weeks or less, upon once daily repeated dosing of the present pharmaceutical composition comprising minocycline.

The term “Cₘₐₓ” refers to the maximum concentration; used herein with respect to the plasma concentration or the interstitial fluid (ISF) (dermal) concentration, which may also be identified as “Cₘₐₓₚ” and “C_{maxISE}”, respectively.

The term “Cave” refers to the average concentration for a time period (e.g., 24 hours or a 1 day); and is used herein with respect to the plasma concentration or the interstitial fluid (ISE) (dermal) concentration, which may also be identified as “C_{avgp}” and “C_{avgISF}”, respectively.

The term “Cₘᵢₙ” refers to the minimum concentration over a time period (e.g., 24 hours or a 1 day); and is used herein with respect to the plasma concentration or the interstitial fluid (ISE) (dermal) concentration, which may also be identified as “Cₘᵢₙₚ” and “Cₘᵢₙₛᵣ”, respectively.

The term “Tₘₐₓ” refers to the time to reach the maximum concentration (Cₘₐₓ) for a time period (e.g., 24 hours or a 1 day); and is used herein with respect to the plasma concentration or the interstitial fluid (ISF) (dermal) concentration,

The term “AUC” or “AUC₀₋₁” refers to the area under the concentration-time curve with respect to the plasma or interstitial fluid (ISF) (dermal) concentration as disclosed herein; or the drug (minocycline or doxycycline) exposure from time zero to time t, where “t” is the last. sampling time point with measurable drug concentration.

### Embodiments

Oral tetracycline compounds are known for treating inflammatory skin conditions like rosacea. However, conventional oral tetracycline compounds require higher doses with repeated administration that cause a higher degree of fluctuation and/or variations of the plasma concentrations, thereby compromising the treatment regimen with increased side effects. Having a treatment option with a reduced dose; less fluctuation between peak (Cₘₐₓ) and (Cₘᵢₙ) plasma concentrations with an earlier onset of effective and steadier ISF (dermal) concentrations, is a long-felt need.

The presently disclosed invention is based, in part, on the surprising discoveries disclosed herein. Notably, the presently disclosed formulation of minocycline exhibits effective treatment results after only 2 weeks of daily oral administration without or with fewer undesirable side effects caused by high plasma concentrations.

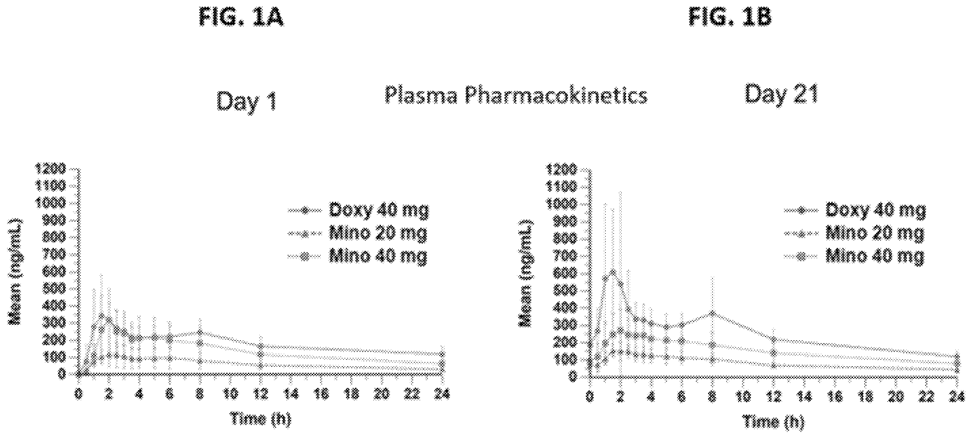

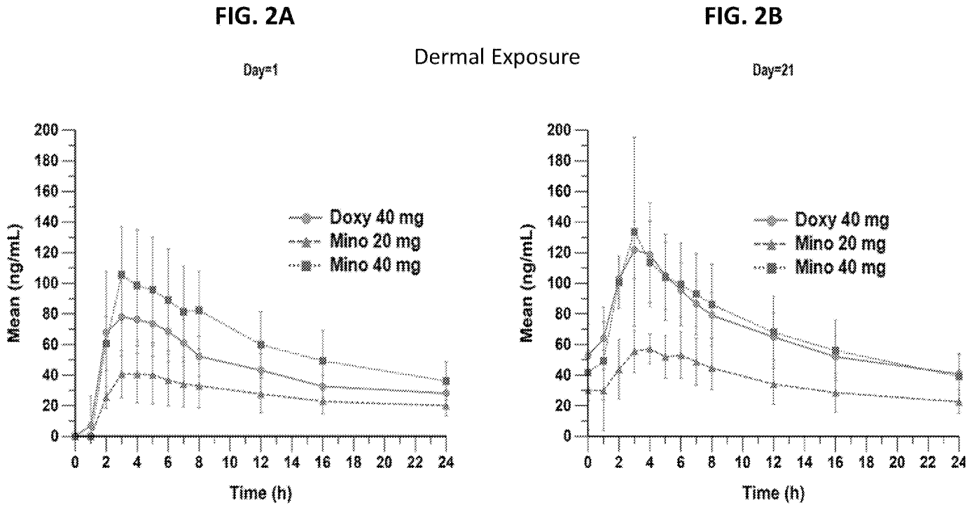

With reference to FIGS. 1A and 1B, the systemic exposure (i.e., plasma concentration) of the minocycline formulation (40 mg and 20 mg) shows minimal fluctuation from the first day (Day 1) of administration. This decreased fluctuation in plasma concentration for minocycline is unlike doxycycline at 40 mg, which after oral administration for 3 weeks, slowly accumulated in the plasma by Day 21. Additionally, with reference to FIGS. 2A and 2B, equilibration of the dermal mean concentration (interstitial fluid, ISF) of the minocycline formulation occurs rapidly (Day 1) and moderately increases (Day 21) with continued daily administration, whereas doxycycline is slower to equilibrate and reach steady state. With reference to Table 2, at Day 21, doxycycline is found at higher concentrations (Cₘₐₓ and C_{avg}) in the plasma than in the dermis (ISF).

With continued reference to FIGS. 1A, 1B, 2A, 2B, and Table 2, the present application relates to a method of treating or a pharmaceutical composition for the use in treating moderate to severe rosacea in a subject suffering therefrom. Advantageously, orally administrating minocycline formulated with IR and ER portions as disclosed herein, results in the minocycline rapidly reaching the interstitial fluid (ISF) (i.e., the dermis), the site of the rosacea where it is needed to treat the rosacea. Notably, the dermal mean concentration of the minocycline quickly reaches a steady state and shows a moderate increase with daily administration over time, whereas the plasma concentration of minocycline is lower than the dermal mean concentration and does not fluctuate more than about 20% with daily administration. In other words, the minocycline formulated with IR and ER portions as disclosed herein, more rapidly targets the rosacea to be treated in the ISF with fewer or no side effects due to its lower and steady plasma concentrations.

In some embodiments, the pharmaceutical composition is formulated with 40 mg minocycline with IR and ER portions as disclosed herein, wherein after daily administration of the formulated minocycline for 2, 4, 8, 12, or 16 weeks, both the plasma concentration and the dermal mean (ISF) concentrations of minocycline do not increase by more than about 20% relative to Day 1.

In some embodiments, the pharmaceutical composition is formulated with 40 mg minocycline with IR and ER portions as disclosed herein, wherein after daily administration of the formulated minocycline for 3 weeks (21 days), both the plasma concentration and the dermal mean (ISF) concentration of minocycline do not increase by more than about 20% on Day 21 compared to Day 1.

In some embodiments, the pharmaceutical composition is formulated with 40 mg minocycline with IR and ER portions as disclosed herein, wherein after daily administration of the formulated minocycline to a subject for 3 weeks (21 days), the interstitial fluid (ISF) AUC₀₋₂₄ and Cₘₐₓ in the subject do not increase by more than 20% on Day 21 compared to Day 1.

In some embodiments, the pharmaceutical composition is formulated with 40 mg minocycline with IR and ER portions as disclosed herein, wherein after daily administration of the formulated minocycline to a subject for 3 weeks (21 days), the interstitial fluid (ISF) AUC₀₋₂₄ and Cₘₐₓ in the subject on Day 1 is at least 80% of the ISF AUC₀₋₂₄ and Cₘₐₓ on Day 21.

In some embodiments, the pharmaceutical composition is formulated with 40 mg minocycline with IR and ER portions as disclosed herein, wherein after daily administration of the formulated minocycline to a subject for 3 weeks (21 days), the interstitial fluid (ISF) AUC₀₋₂₄ and Cₘₐₓ in the subject do not increase by more than 15% on Day 21 compared to Day 1.

In some embodiments, the pharmaceutical composition is formulated with 40 mg minocycline with IR and ER portions as disclosed herein, wherein after daily administration of the formulated minocycline to a subject for 3 weeks (21 days), the interstitial fluid (ISF) AUC₀₋₂₄ and Cₘₐₓ in the subject do not increase by more than 20% on Day 21 compared to Day 1 and the ISF AUC₀₋₂₄ and Cₘₐₓ are each less than the respective plasma AUC₀₋₂₄ and Cₘₐₓ values on Days 1 and 21.

In some embodiments, the pharmaceutical composition is formulated with 40 mg minocycline with IR and ER portions as disclosed herein, wherein after daily administration of the formulated minocycline to a subject for 3 weeks, the interstitial fluid (ISF) AUC₀₋₂₄ and Cₘₐₓ in the subject do not increase by more than 20% on Day 21 compared to Day 1, and the plasma AUC₀₋₂₄ and Cₘₐₓ values on Day 21 differ from the respective values on Day 1 by 15% or less. Accordingly, the plasma concentrations of the disclosed minocycline have low fluctuation. In comparison, the disclosed minocycline has decreased fluctuation compared to the plasma concentration fluctuations observed for doxycycline on Days 1 and 21 (Table 2).

Without being bound to any theory, the improved pharmacokinetics and efficacy of the lower dose minocycline formulation is the result of an optimal mix of IR/ER and a higher tissue penetration of the minocycline due to its lower protein binding and higher lipophilicity of the minocycline composition. Furthermore, the improved pharmacokinetics and efficacy of the presently disclosed formulation of minocycline allow for lower, body weight-independent doses compared to the currently available doses of minocycline which are provided at mg/kg body weight of the subject. For example, Solodyn® requires a body weight-dependent dosage, and is administered at 45 to 200 mg per day. Notably, the minimal dose of Solodyn® is 45 mg per day for the lowest permitted body weight of 45 kg.

Accordingly, the present application relates to a method of treating or the use of an oral pharmaceutical composition for treating moderate to severe rosacea with a therapeutically effective amount of 20 to 40 mg minocycline in a formulation as disclosed herein and administered on a daily basis for at least 2 weeks. The treatment includes once daily oral administration of the minocycline composition as disclosed herein for a period of at least 2 weeks. The treatment period may continue on a daily basis as needed. The treatment period may be any of 2, 3, 4, 5, 6, 7, 8, 9, 10, 11, 12, 13, 14, 15, or 16 weeks, but is not limited to only full week increments. As such, the treatment period is at least 14 days (2 weeks) and may continue indefinitely as needed. For example, the treatment period may be 14 days up to any of 112 days or more.

In some embodiments, the oral pharmaceutical composition comprising minocycline for use in treating moderate to severe rosacea is a composition of 20 mg to 40 mg minocycline hydrochloride formulated in an immediate release (IR) portion and an extended release (ER) portion, with an IR to ER ratio ranging from 10:90 to 90:10. The IR to ER ratio may be about 10:90, about 20:80, about 25:75, about 30:70, about 40:60, about 50:50, about 60:40, about 70:30, about 75:25, about 80:20, or about 90:10. In other embodiments, the pharmaceutical composition comprises an immediate release (IR) portion and an extended release (ER) portion with an IR to ER ratio of about 50:50, about 40:60, about 30:70, about 25:75, or about 20:80. Typically, the oral pharmaceutical composition is about 40 mg minocycline hydrochloride formulated in and IR to ER ratio of 25:75.

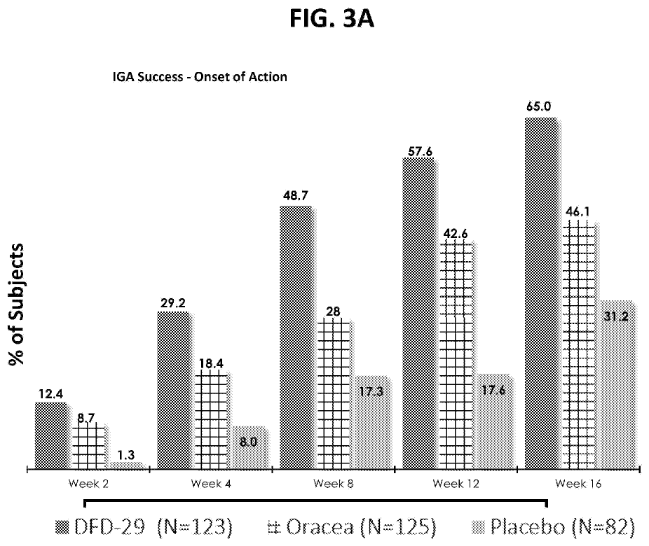

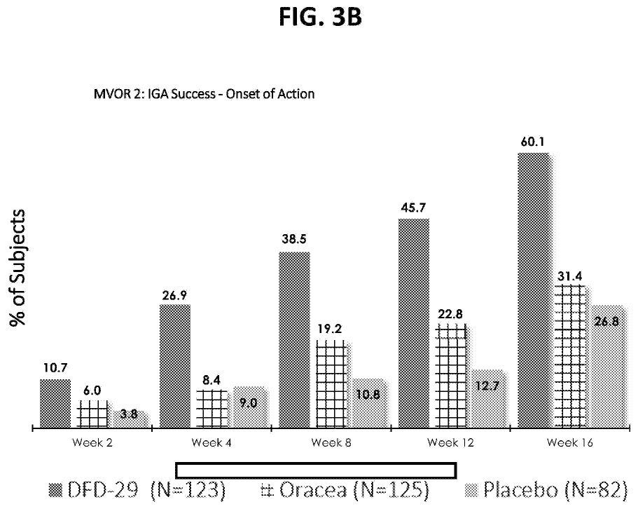

With reference to Tables 3A-3D and FIGS. 3A and 3B, a method of treating or a pharmaceutical composition for the use in treating rosacea in a subject having an Investigator's Global Assessment (IGA) score of 3 (moderate) or 4 (severe) rosacea, and orally administering the pharmaceutical composition as disclosed herein formulated with about 40 mg of minocycline to the subject on a daily basis, wherein after at least 2 weeks of daily administration, the reduction in the severity of the rosacea comprises a reduction in IGA by at least one grade, In some embodiments, the reduction in the severity of the rosacea comprises a reduction in IGA by at least two grades after 2 weeks of daily administration. In some embodiments, the pharmaceutical composition is formulated with 40 mg minocycline with IR and ER portions as disclosed herein. In other embodiments, the reduction in the severity of the rosacea comprises a reduction in IGA by at least two grades after 2 weeks, 4 weeks, 8 weeks, 12 weeks, or 16 weeks of daily administration. In still other embodiments, the IGA score is reduced from 3 or 4 to 0 or 1 after 2 weeks, 4 weeks, 8 weeks, 12 weeks, or 16 weeks of daily administration.

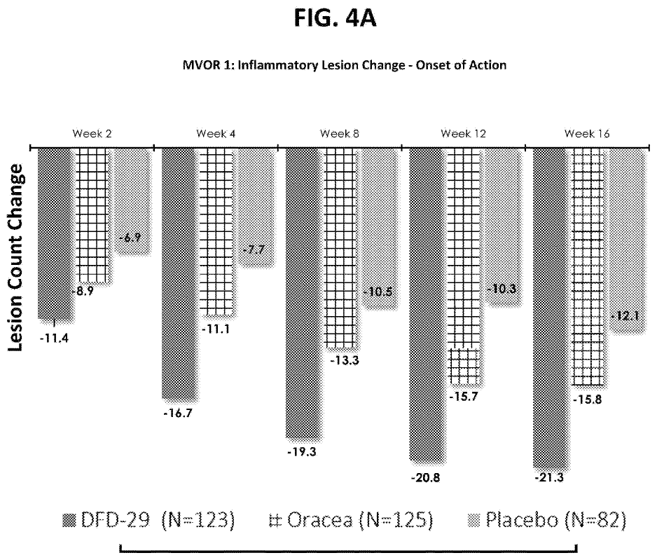

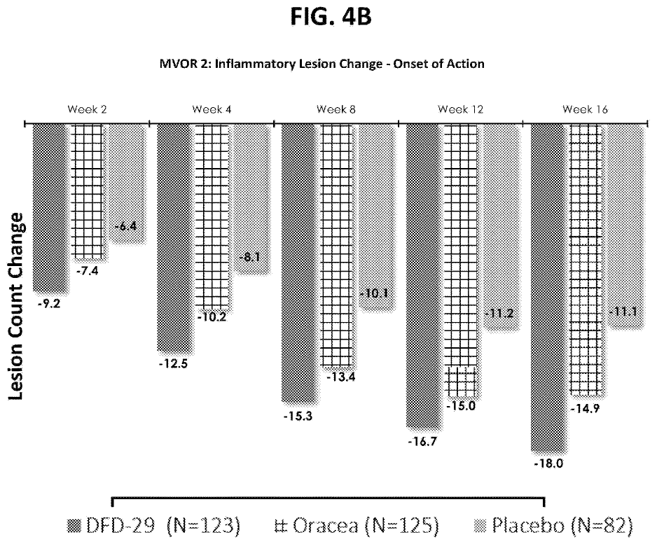

With reference to Tables 4A-4F and FIGS. 4A and 4B, a method of treating or a pharmaceutical composition for the use in treating moderate to severe rosacea in a subject suffering therefrom, includes orally administering the pharmaceutical composition as disclosed herein formulated with about 40 mg of minocycline to the subject on a daily basis, wherein upon orally administering the pharmaceutical composition for at least 2 weeks, the severity of the rosacea is reduced with a decrease in lesion count as compared to the lesion count before administering the pharmaceutical composition. For example, after oral administration on a daily basis of the presently disclosed minocycline composition to a subject having moderate to severe rosacea (e.g., having at least 10 lesions), the lesion count decreases by at least 20% after 2 weeks. In some embodiments, the pharmaceutical composition is formulated with 40 mg minocycline with IR and ER portions as disclosed herein. In other embodiments, the severity of the rosacea is reduced with a decrease in lesion count by at least 25%, 30%, 40%, 45% or 50% in 2 weeks. In some embodiments, the severity of the rosacea is reduced with a decrease in lesion count by at least 25%, 30%, 40%, 45% or 50% in 3 weeks. In some embodiments, the severity of the rosacea is reduced with a decrease in lesion count by at least 25%, 30%, 40%, 45%, 50%, 55%, or 60% in 4 weeks. In some embodiments, the severity of the rosacea is reduced with a decrease in lesion count by at least 25%, 30%, 40%, 45%, 50%, 55%, or 60% in 5 weeks. In some embodiments, the severity of the rosacea is reduced with a decrease in lesion count by at least 25%, 30%, 40%, 45%, 50%, 55%, or 60% in 6 weeks. In some embodiments, the severity of the rosacea is reduced with a decrease in lesion count by at least 25%, 30%, 40%, 45%, 50%, 55%, 60%, 65%, or 70% in 7 weeks. In some embodiments, the severity of the rosacea is reduced with a decrease in lesion count by at least 25%, 30%, 40%, 45%, 50%, 55%, 60%, 65%, 70%, 75%, 76%, 77%, 78%, 79%, or 80% in 8 weeks. In some embodiments, the severity of the rosacea is reduced with a decrease in lesion count by at least 25%, 30%, 40%, 45%, 50%, 55%, 60%, 65%, 70%, 75%, 76%, 77%, 78%, 79%, or 80% in 9 weeks. In some embodiments, the severity of the rosacea is reduced with a decrease in lesion count by at least 25%, 30%, 40%, 45%, 50%, 55%, 60%, 65%, 70%, 75%, 76%, 77%, 78%, 79%, or 80% in 10 weeks. In some embodiments, the severity of the rosacea is reduced with a decrease in lesion count by at least 25%, 30%, 40%, 45%, 50%, 55%, 60%, 65%, 70%, 75%, 76%, 77%, 78%, 79%, or 80% in 11 weeks. In some embodiments, the severity of the rosacea is reduced with a decrease in lesion count by at least 25%, 30%, 40%, 45%, 50%, 55%, 60%, 65%, 70%, 70%, 75%, 76%, 77%, 78%, 79%, 80%, 81%, or 82% in 12 weeks. In some embodiments, the severity of the rosacea is reduced with a decrease in lesion count by at least 25%, 30%, 40%, 45%, 50%, 55%, 60%, 65%, 70%, 75%, 76%, 77%, 78%, 79%, 80%, 81%, or 82% in 13 weeks. In some embodiments, the severity of the rosacea is reduced with a decrease in lesion count by at least 25%, 30%, 40%, 45%, 50%, 55%, 60%, 65%, 70%, 75%, 76%, 77%, 78%, 79%, 80%, 81%, or 82% in 14 weeks. In some embodiments, the severity of the rosacea is reduced with a decrease in lesion count by at least 25%, 30%, 40%, 45%, 50%, 55%, 60%, 65%, 70%, 75%, 76%, 77%, 78%, 79%, 80%, 81%, or 82% in 15 weeks. In some embodiments, the severity of the rosacea is reduced with a decrease in lesion count by at least 25%, 30%, 40%, 45%, 50%, 55%, 60%, 65%, 70%, 75%, 76%, 77%, 78%, 79%, 80%, 81%, 82%, 83%, 84%, 85%, or 86% in 16 weeks.

In some embodiments, a method of treating or a pharmaceutical composition for the use in treating rosacea in a subject having at least 15 facial rosacea lesions, wherein after at least 2 weeks of daily oral administration to the subject with 40 mg minocycline composition as disclosed herein, the number of facial rosacea lesions is reduced by at least 20%. In some embodiments, the pharmaceutical composition is formulated with 40 mg minocycline with IR and ER portions as disclosed herein. In some embodiments, the number of facial rosacea lesions is reduced by at least 20%, 25%, 30%, 40%, 45% or 50% after 2 weeks of daily administration. In some embodiments, the number of facial rosacea lesions is reduced by at least 20%, 25%, 30%, 40%, 45%, 50%, 55%, or 60% after 4 weeks of daily administration. In some embodiments, the number of facial rosacea lesions is reduced by at least 20%, 25%, 30%, 40%, 45%, 50%, 55%, 60%, 65%, 70%, 75%, 76%, or 77% after 8 weeks of daily administration. In some embodiments, the number of facial rosacea lesions is reduced by at least 20%, 25%, 30%, 40%, 45%, 50%, 55%, 60%, 65%, 70%, 75%, 80%, 81%, or 82% after 12 weeks of daily administration. In some embodiments, the number of facial rosacea lesions is reduced by at least 20%, 25%, 30%, 40%, 45%, 50%, 55%, 60%, 65%, 70%, 75%, 80%, 85%, or 86% after 16 weeks of daily administration,

The presently disclosed minocycline composition is effective at treating moderate to severe rosacea in a subject of any skin color. That is, the minocycline composition disclosed herein is effective at treating rosacea in a subject having Fitzpatrick skin type of I, II, III, IV, V, or VI.

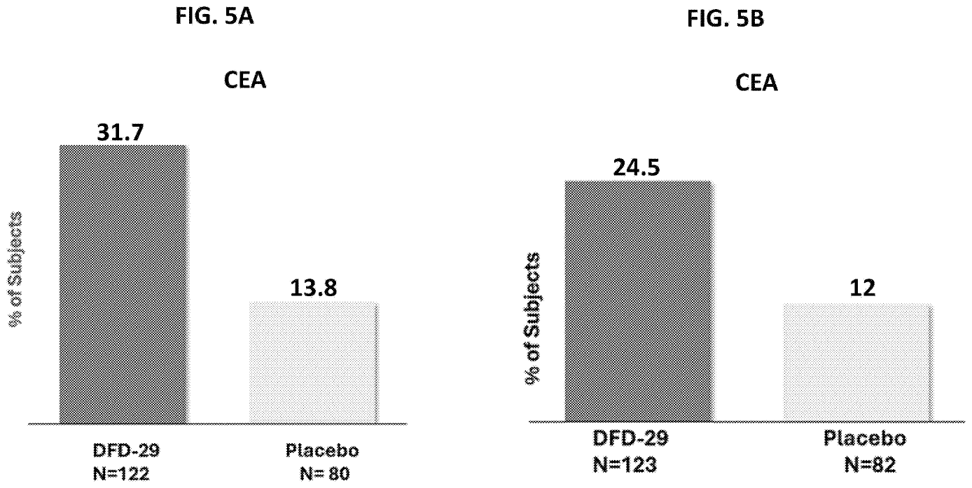

With reference to Tables 5A-5D and FIGS. 5A and 5B, a method of treating or a pharmaceutical composition for the use in treating moderate to severe rosacea in a subject suffering therefrom, includes orally administering the pharmaceutical composition comprising about 40 mg of minocycline to the subject on a daily basis, wherein upon orally administering the pharmaceutical composition for at least 2 weeks, the reduction in the severity of the rosacea comprises a reduction in Clinicians Erythema Assessment (CEA) score. In some embodiments, the pharmaceutical composition is formulated with 40 mg minocycline with IR and ER portions as disclosed herein. In some embodiments, the CEA score is reduced by at least 2 grades. In other embodiments, the CEA score is reduced by at least 2 grades in 2, 4, 8, 12, or 16 weeks.

With reference to Tables 6A-6D, method of treating or a pharmaceutical composition for the use in treating moderate to severe rosacea in a subject suffering therefrom, includes orally administering the pharmaceutical composition comprising about 20 to 40 mg of minocycline to the subject on a daily basis, wherein upon orally administering the pharmaceutical composition for at least 2 weeks, results in improved quality of life as indicated by a decrease in a Rosacea Quality of Life score and/or a decrease in a Dermatology Life Quality Index score. In some embodiments, the pharmaceutical composition is formulated with 40 mg minocycline with IR and ER portions as disclosed herein, and the decrease in a Rosacea Quality of Life score or decrease in Dermatology Life Quality Index score occurs after at least 4, 8, 12, or 16 weeks of daily administration of the pharmaceutical composition.

Accordingly, the present application relates to a pharmaceutical composition comprising formulated minocycline for effectively treating rosacea with lower fluctuation index, leading to lesser incidence of side effects with effective treatment (e.g., a significant reduction in the number of lesions) as early as 2 weeks of daily administration.

In an aspect of the above embodiments, the method of treating rosacea by administering an oral pharmaceutical composition comprising a reduced dose of minocycline to a subject in need thereof, wherein said rosacea is a papulopustular rosacea, acne rosacea, an erythematotelangiectatic rosacea, a phymatous rosacea, an ocular rosacea, an acne rosacea, a pyoderma faciale, a rosacea conglobate, a mild rosacea, a moderate rosacea, a severe rosacea, a mild to moderate rosacea, or a moderate to severe rosacea.

In another aspect of the above embodiments, the method of treating rosacea by administering an oral pharmaceutical composition comprising a reduced dose of minocycline to a subject in need thereof, wherein said rosacea is characterized by papules, pustules and/or nodules.

In an aspect of the above embodiments, the method of treating rosacea by administering a reduced dose of minocycline, wherein said administration significantly reduced the severity of rosacea as compared to the severity of rosacea before the treatment.

In an aspect of the above embodiments, the method of treating rosacea by administering a reduced dose of minocycline, wherein said administration significantly reduced the severity of rosacea as compared to the severity of rosacea before the treatment, and wherein said severity of the rosacea is assessed using IGA scale.

In an aspect of the above embodiments, the method of treating rosacea by administering a reduced dose of minocycline, wherein said administration significantly reduced the severity of rosacea as compared to the severity of rosacea before the treatment, and wherein said severity of the rosacea is assessed counting a number of inflammatory lesions.

In an aspect of the above embodiments, the method of treating rosacea by administering a reduced dose of minocycline, wherein said administration significantly reduces the number of inflammatory lesions compared to the number of inflammatory lesions before the treatment.

In an aspect of the above embodiments, the pharmaceutical composition of present application is administered once daily.

In another aspect of the above embodiments, the pharmaceutical composition of present application is with or without food.

In an aspect of the above embodiments, the present application relates to a method of treating rosacea by administering a pharmaceutical composition comprising a therapeutic effective amount of 20 mg to 40 mg minocycline as disclosed herein, to a subject in need thereof, wherein said composition is administered orally in the form of oral tablets, capsules, pills, minitablets, pellets, granules, powder, suspension, syrup and the like.

In an embodiment, the present application relates to a method of treating an inflammatory skin condition by administering a pharmaceutical composition comprising 20 mg to 40 mg minocycline as disclosed herein, or a pharmaceutically acceptable salt thereof to a subject in need thereof, and measuring plasma concentration and/or interstitial fluid concentration of minocycline in the subject.

As will be recognized by one of ordinary skill in the art, depending on the circumstances, plasma concentration and/or interstitial fluid concentration of minocycline can be measured daily, more than daily, or less than daily during an entire course of treatment or a portion of a course of treatment of the subject. In some embodiments, for example, the subject can be measured for 1, 2, 3, 4, 5, 6, 7, 8, 9, 10, 11, 12, 13, 14, 15, or 16 weeks.

In an embodiment, the present application relates to a pharmaceutical composition comprising minocycline for treating an inflammatory skin condition in a subject in need thereof, wherein said composition is in the form of oral tablets, capsules, pills, minitablets, pellets, granules, powder, suspension or syrup.

In an embodiment, the present application relates to a pharmaceutical composition comprising minocycline for treating rosacea in a subject in need thereof, wherein said composition is in the form of oral tablets, capsules, pills, minitablets, pellets, granules, powder, suspension or syrup.

In an embodiment, the present application relates to a pharmaceutical composition comprising minocycline for treating acne in a subject in need thereof, wherein said composition is in the form of oral tablets, capsules, pills, minitablets, pellets, granules, powder, suspension or syrup.

In an aspect of the above embodiments, the immediate release (IR) and/or extended release (ER) portions are present in the form of a granule, pellet, bead, spherule, mini tablet, powder and the like or mixtures thereof.

In an embodiment, the present application relates to a process of preparing pharmaceutical composition comprising a therapeutically effective amount of minocycline.

In another aspect of the above embodiments, the process involves conventional methods to prepare oral pharmaceutical composition, that includes, but not limited to, wet or dry granulation, using fluidized bed granulator or high shear mixer granulator, direct compression, extrusion-spheronization, melt granulation/extrusion, spray-drying, spray-congealing, freeze-drying, or any other conventional process known in the art.

In an aspect of the above embodiments, the process involves coating or layering of minocycline over inert cores with coating or layering materials comprising minocycline and/or other suitable pharmaceutically acceptable excipients like binders, plasticizers or disintegrants over the inert cores.

The process of coating or layering includes any method known in the art such as, but not limited to, by spraying a suspension or dispersion of said coating material comprising minocycline, in a conventional coating pan or fluidized bed equipment (such as a Wurster or Glatt) followed by drying of cores. Alternatively, said coating materials may also be applied by powder-coating, wherein the cores are maintained in a sticky state, a mixture of coating material is added continuously or periodically so as to adhere to the sticky cores, followed by drying of coated cores when desired coating is achieved.

The “inert core” as used herein, refers to a pharmaceutically acceptable inert substrate which is routinely used in formulation art, that includes, but not limited to, powder or a multiparticulate such as a granule, a pellet, a bead, a spherule, a beadlet, a microcapsule, a millisphere, a nanocapsule, a nanosphere, a microsphere or a minitablet, which comprises at least one pharmaceutically acceptable excipient selected from the group comprising of water soluble, water insoluble, water swellable or water non swellable material such as starch, sugar, microcrystalline cellulose, vegetable gums, waxes, and the like.

The inert cores may also be prepared with the techniques known to a person skilled in the art, such as, wet granulation, dry granulation, or extrusion--spheronization and the like. The inert cores have a size of diameter in the range of about 125 to about 600 microns.

Suitable solvent(s) used in the preparation of minocycline solution are selected from, but not limited to, water, methanol, ethanol, n-propanol, isopropanol, dichloromethane, acetone, absolute alcohol and the like or mixtures thereof.

Suitable examples of binder(s) that may be used in the present application include, but are not limited to, methyl cellulose, hydroxy propyl cellulose, hydroxy propyl methyl cellulose, polyvinyl pyrrolidone, polyvinyl alcohol, propylene glycol, pre gelatinized starch, oxide such as polyethylene oxide and the like or mixtures thereof. The binders may be combination of two or more, such as hydroxy propyl cellulose and hydroxy propyl methyl cellulose. The binders used in the present application have a viscosity from about 5 centipoise to about 15 centipoise.

In another aspect of the above embodiments, the extended release (ER) portion are prepared by coating the immediate release (IR) portion with one or more release modifying polymers.

In another aspect of the above embodiments, the present application relates to a process of preparing pharmaceutical composition of minocycline comprising extended release (ER) portion, wherein the ER coating layer has a thickness of not more than 200 μm.

Suitable examples of release modifying polymers that may be used in the present application include, but are not limited to, unsubstituted alkyl celluloses or cellulose ethers like ethyl cellulose; and substituted alkyl celluloses or cellulose ethers like hydroxy alkyl celluloses and carboxy alkyl celluloses such as hydroxy ethyl cellulose, hydroxy propyl cellulose, hydroxy propyl methyl cellulose, carboxy methyl ethyl cellulose and carboxy methyl cellulose: acrylic and methacrylic acid polymers and copolymers such as methyl methacrylate, ethoxy ethyl methacrylates, ethyl acrylate, amino alkyl methacrylate copolymer, poly(acrylic acid), poly(methacrylic acid), polyacrylamide and glycidyl methacrylate copolymers; polyalkylene oxides such as polyethylene oxide and polypropylene oxide and copolymers of ethylene oxide and propylene oxide; poly vinyl alcohols, gums, synthetic resins and the like or mixtures thereof. The release modifying polymers may be present in amounts ranging from about 5% to about 45% w/w of the composition.

In another embodiment, the pharmaceutical composition of minocycline of present application may comprises one or more pharmaceutically acceptable excipient(s) selected from lubricants, glidants, anti-tacking agents, plasticizers, disintegrants or opacifying agents and the like or mixtures thereof.

The lubricant, glidant or anti-tacking agent may be used interchangeably in the composition of the present application and are selected from, but not limited to, metallic stearates such as magnesium stearate, calcium stearate, zinc stearate; stearic acid, hydrogenated vegetable oil, hydrogenated castor oil, glyceryl palmitostearate, glyceryl behenate, polyethylene glycols, corn starch, sodium stearyl fumarate, sodium benzoate, mineral oil, talc, colloidal silicon dioxide, magnesium trisilicate, powdered cellulose, starch, tribasic calcium phosphate and the like or mixtures thereof. The amount of such agents may range from about 0.1% w/w to about 10% w/w of the composition.

The plasticizer used in the pharmaceutical composition of the present application may be used in the coating layer to increase the flexibility and strength of the coat/layer, and suitable plasticizer may be selected from, but not limited to, propylene glycol, polyethylene glycol, triethyl citrate, acetyl triethyl citrate, diethyl phthalate, dibutyl phthalate, dibutyl sebacate, tributyl citrate or mixtures thereof. The plasticizer may be present in amounts ranging from about 0.1% to about 20% w/w of the composition.

The disintegrant used in the pharmaceutical composition of the present application may be selected from, but not limited to, crospovidone, sodium starch glycolate, croscarmellose sodium, croscarmellose potassium, croscarmellose calcium, carboxymethylcellulose, pregelatinized starch, carboxymethyl starch and the like or mixtures thereof. The disintegrant may be present in amount from 1% to 20% by weight of the composition.

In an aspect of the above embodiments, the present process of preparing pharmaceutical. composition comprising about 20 mg to about 40 mg dose of minocycline comprises steps of: (i) preparing an immediate release (IR) portion; and/or (ii) preparing an extended release (ER) portion; (iii) combining the IR and/or ER portions with one or more pharmaceutically acceptable excipient(s); and (iv) filling the mixture of (iii) into a capsule or compressed into a tablet.

In an aspect of the above embodiments, the present process of preparing pharmaceutical composition comprising 40 mg dose of minocycline comprises steps of: (i) preparing an immediate release (IR) portion; and/or (ii) preparing an extended release (ER) portion; (iii) combining the IR and/or ER portions in a ratio of about 25:75 with one or more pharmaceutically acceptable excipient(s); and (iv) filling the mixture of (iii) into a capsule or compressed into a tablet.

In an embodiment, the pharmaceutical composition comprising minocycline can also be co-administered (simultaneously or sequentially) with one or more pharmaceutical agents of value in the form of commercially available dosage forms or which can be developed in a suitable pharmaceutically acceptable dosage forms for treating an inflammatory skin condition or related disease conditions. In another embodiment, the pharmaceutical composition comprising minocycline can be subjected to dissolution studies in 500 ml of pH 2.1 simulated gastric fluid, pH 4.5 acetate buffer and pH 6.8 phosphate buffer, with USP Type I apparatus at a speed of 100 rpm and 37° C. till 3 hours.

Examples of the pharmaceutical agents that can be co-administered are selected from, but not limited to, systemic and topical antibiotics like tetracycline, minocycline, doxycycline, metronidazole, erythromycin and clindamycin; retinoids like tretinoin (vitamin A or retinoic acid), isotretinoin (13-cis-retinoic acid), and acitretin; benzoyl peroxide; ivermectin; or azelaic acid; and the like, including mixtures thereof.

The present application is further illustrated by the examples which are provided merely to be exemplary of the pharmaceutical composition described above and do not limit the scope of the application. Certain modifications and equivalents will be apparent to those skilled in the art and are intended to be included within the scope of the present application. The present invention is illustrated below by reference to the following examples. However, one skilled in the art will appreciate that the specific methods and results discussed are merely illustrative of the present invention, and not to be construed as limiting the application. The following examples may include compilations of data that are representative of data gathered at various times during the course of development and experimentation related to the present invention.

### Examples

Example 1. Pharmaceutical compositions used in the following examples include ORACEA® and minocycline at 20 mg and 40 mg. Oracea capsules contain doxycycline 40 mg, hereafter referred to as “Oracea” for when administered in the U.S. and “Oraycea” when administered in Europe,

The minocycline hydrochloride (HCl) Extended Release (ER) capsules at 20 mg and 40 mg (herein referred to as “DFD-29 (20 mg)” and “DFD-29 (40 mg)”) were previously disclosed in US 2019/0209500, the entire contents of which are herein incorporated by reference. The ER minocycline capsule is formulated with 25% (5 or 10 mg) of the minocycline as immediate release (IR) and 75% (15 or 30 mg) of the minocycline is formulated as extended release (ER).

In brief, minocycline HCl having an extended release (ER) portion and an immediate release (IR) portion, was prepared according to the following steps:
a. Mixing minocycline hydrochloride, hydroxy propyl methyl cellulose, hydroxy propyl cellulose, polyethylene glycol and talc in water and layered onto inert core to obtain drug loaded pellets.
b. A barrier coating solution was prepared by dissolving hydroxy propyl methyl cellulose, in water followed by adding polyethylene glycol and tale with stirring.
c. A release modifying coating solution was prepared by dissolving the required amount of ethyl cellulose and hydroxy propyl methyl cellulose in isopropyl alcohol-water solvent system, followed by adding triethyl citrate, and talc.
d. An immediate release (IR) portion was prepared by coating the barrier coating solution as prepared in step (c) onto a drug loaded portion of step (a).
e. An extended release (ER) portion was prepared by coating the release modifying coating solution as prepared in step (c) onto the drug loaded portion of step (a), followed by an outer top or seal coating.
f. A required quantity of the outer top coated portions and the barrier coated portions were mixed with talc and filled into a required size of empty hard gelatine capsule shells and packaged in a suitable pharmaceutical storage bottle.

Example 2. Pharmacokinetic parameters. The pharmacokinetic endpoints determined for daily treatments of DFD-29 (minocycline HCl) Extended Release Capsules 20 mg and 40 mg and ORAYCEA (doxycycline) Capsules 40 mg on Day 1 and Day 21 are presented below in Table 2.

TABLE 2
Pharmacokinetic endpoints after treatment on Day 1 and Day 21

| | | Day 1 (Visit 2) | | | | Day 21 (Visit 3) | | | |
|---|---|---|---|---|---|---|---|---|---|
| Parameters | Samples | N | Mean | SD | CV | N | Mean | SD | CV |

DFD-29 (minocycline HCl) Extended Release Capsules 20 mg

| | | | | | | | | | |
|---|---|---|---|---|---|---|---|---|---|
| AUC₀₋₂₄ (ng/mL × h) | ISF | 8 | 589.89 | 231.34 | 39.22 | 7 | 877.82 | 276.97 | 31.55 |
| | PLASMA | 8 | 1412.31 | 757.07 | 53.61 | 7 | 1953.92 | 645.83 | 33.05 |
| Cₘₐₓ (ng/mL) | ISF | 8 | 42.94 | 16.34 | 38.05 | 5 | 60.64 | 10.75 | 17.73 |
| | PLASMA | 8 | 130.68 | 66.49 | 50.88 | 5 | 164.46 | 70.87 | 43.09 |
| Tₘₐₓ (h) | ISF | 8 | 3.63 | 1.06 | 29.26 | 5 | 3.80 | 0.84 | 22.02 |
| | PLASMA | 8 | 2.19 | 1.62 | 74.25 | 5 | 1.90 | 0.96 | 50.62 |
| Cₘᵢₙ (ng/mL) | ISF | 8 | 1.19 | 1.71 | 143.17 | 5 | 16.37 | 8.63 | 52.68 |
| | PLASMA | 8 | 4.01 | 1.83 | 45.60 | 5 | 35.11 | 15.36 | 43.73 |
| C_{avg} (ng/mL) | ISF | 8 | 26.26 | 9.75 | 37.14 | 5 | 37.14 | 9.76 | 26.28 |
| | PLASMA | 8 | 73.53 | 37.74 | 51.32 | 5 | 95.76 | 34.63 | 36.16 |

DFD-29 (minocycline HCl) Extended Release Capsules 40 mg

| | | | | | | | | | |
|---|---|---|---|---|---|---|---|---|---|
| AUC₀₋₂₄ (ng/mL × h) | ISF | 8 | 1412.29 | 484.82 | 34.33 | 8 | 1604.77 | 454.26 | 28.31 |
| | PLASMA | 8 | 3549.64 | 1438.64 | 40.53 | 8 | 3957.62 | 1098.95 | 27.77 |
| Cₘₐₓ (ng/mL) | ISF | 8 | 109.68 | 32.83 | 29.93 | 8 | 125.69 | 55.06 | 43.80 |
| | PLASMA | 8 | 382.83 | 188.74 | 49.30 | 8 | 337.74 | 95.45 | 28.26 |
| Tₘₐₓ (h) | ISF | 8 | 3.38 | 0.74 | 22.05 | 8 | 3.13 | 1.36 | 43.40 |
| | PLASMA | 8 | 1.88 | 0.52 | 27.60 | 8 | 1.69 | 0.84 | 49.93 |
| Cₘᵢₙ (ng/mL) | ISF | 8 | 2.77 | 1.89 | 68.11 | 8 | 39.94 | 16.95 | 42.44 |
| | PLASMA | 8 | 3.29 | 1.94 | 58.98 | 8 | 96.96 | 47.93 | 49.43 |
| C_{avg} (ng/mL) | ISF | 8 | 63.61 | 20.81 | 32.71 | 8 | 76.86 | 21.39 | 27.82 |
| | PLASMA | 8 | 187.43 | 77.49 | 41.35 | 8 | 210.01 | 53.13 | 25.03 |

ORAYCEA (doxycycline) Capsule 40 mg

| | | | | | | | | | |
|---|---|---|---|---|---|---|---|---|---|
| AUC₀₋₂₄ (ng/mL × h) | ISF | 7 | 1088.55 | 261.79 | 24.05 | 8 | 1573.63 | 380.98 | 24.21 |
| | PLASMA | 8 | 4377.52 | 1232.31 | 28.15 | 8 | 6074.76 | 1979.89 | 32.59 |
| Cₘₐₓ (ng/mL) | ISF | 7 | 81.52 | 28.01 | 34.37 | 8 | 114.69 | 37.74 | 32.91 |
| | PLASMA | 8 | 405.86 | 170.07 | 41.90 | 8 | 701.85 | 534.15 | 76.11 |
| Tₘₐₓ (h) | ISF | 7 | 4.00 | 1.15 | 28.87 | 8 | 4.00 | 1.51 | 37.80 |
| | PLASMA | 8 | 2.50 | 2.27 | 90.71 | 8 | 1.56 | 0.56 | 36.03 |
| Cₘᵢₙ (ng/mL) | ISF | 7 | 0.80 | 0.92 | 115.01 | 8 | 39.03 | 12.99 | 33.28 |
| | PLASMA | 8 | 5.20 | 5.24 | 100.88 | 8 | 186.32 | 72.60 | 38.96 |
| C_{avg} (ng/mL) | ISF | 7 | 49.54 | 14.66 | 29.58 | 8 | 75.70 | 20.10 | 26.55 |
| | PLASMA | 8 | 211.00 | 75.30 | 35.69 | 8 | 340.38 | 151.88 | 44.62 |

FIGS. 1A, 1B, 2A, and 2B present the mean (+SD) linear dermal (ISF) and plasma concentration-time curves of daily treatment of DFD-29 (minocycline HCl) Extended Release Capsules 20 mg (triangles), 40 mg (squares), and ORAYCEA (doxycycline) Capsules 40 mg (circles), as observed on Day 1 and Day 21. The average AUC₀₋₂₄ and Cₘₐₓ values of DFD-29 (minocycline HCl) Extended Release Capsules 40 mg were higher in dermal mean (ISF) and plasma as compared to DFD-29 (minocycline HCl) Extended Release Capsules 20 mg at both Day 1 and Day 21, while the average Tₘₐₓ values were similar.

Comparing DFD-29 (minocycline HCl) Extended Release Capsules 40 mg with ORAYCEA (doxycycline) Capsules 40 mg, the average AUC₀₋₂₄ and Cₘₐₓ values for DFD-29 (minocycline HCl) Extended Release Capsules 40 mg in dermal (ISF) were significantly higher compared to ORAYCEA (doxycycline) Capsules 40 mg on Day 1. The average AUC₀₋₂₄ and Cₘₐₓ values for DFD-29 (minocycline HCl) Extended Release Capsules 40 mg in dermal (ISF) were comparable to ORAYCEA (doxycycline) Capsules 40 mg on Day 21. The average AUC₀₋₂₄ and Cₘₐₓ values in plasma were comparable between the DFD-29 and ORAYCEA on Day 1, but were significantly lower in DFD-29 (minocycline HCl) Extended Release Capsules 40 mg than ORAYCEA at Day 21. The plasma concentrations as measured by the AUC and Cₘₐₓ for DFD-29 (40 mg) did not increase significantly on Day 21 compared to Day 1. In fact, the plasma AUC for DFD-29 increased by 11.5% and the Cₘₐₓ decreased as measured on Day 21 compared to Day 1. The average value of Tₘₐₓ in DFD-29 (minocycline HCl) Extended Release Capsules 40 mg were similar in plasma as compared to ORAYCEA (doxycycline) Capsules 40 mg at both Day 1 and Day 21, while in the ISF (site of action) DFD-29 had approximately 10-20% lesser average Tₘₐₓ.

Pharmacokinetic conclusions. The systemic exposure (i.e., plasma concentration of minocycline) profile of DFD-29 Extended Release Capsules 40 mg was similar (less than 20% increase) from Day 1 to Day 21, while for Oraycea (doxycycline) Capsules 40 mg, the systemic exposure (i.e., plasma concentration of doxycycline) demonstrated a significant increase (at least about 40%) from Day 1 to Day 21, indicating an accumulation.

The dermal mean ISF concentrations for DFD-29 were similar between Day 1 and Day 21, while for Oraycea, the concentrations increased from Day 1 to Day 21, indicating a delayed equilibration in the dermal ISF for doxycycline, while DFD-29 minocycline shows an early onset of efficacy as early as Week 2 as disclosed in the following examples.

Without being bound to any theory, the faster equilibration of minocycline in the dermal ISF may be a result of the unique formulation (IR/ER) in DFD-29, and minocycline's higher lipophilicity and lower plasma protein binding.

### Example 3. The 40 mg minocycline formulation (DFD-29, Example 1) was comparatively assessed with 40 mg doxycycline capsule with 30 mg immediate release and 10 mg extended release. The comparative assessments were referred to as Minocycline Versus Oracea in Rosacea (MVOR) and were two separate 16-week, multicenter, randomized, parallel-group, double-blind, controlled studies-MVOR-1 and MVOR-2. After assessing eligibility, approximately, 320 subjects at least 18 years old who were diagnosed with moderate (IGA of 3) to severe (IGA of 4) papulopustular rosacea were randomized in a 3:3:2 ratio to Minocycline (40 mg), Doxycycline (40 mg), or Placebo, respectively, for once daily oral administration for a total of 16 weeks. Subject visits were scheduled at Screening, Baseline (Day 1), and Weeks 2, 4, 8, 12, and 16. Clinical assessments of efficacy were conducted based on Investigator's Global Assessment modified scale without erythema (IGA) (Tables 3A, 3B, 3C, 3D and FIGS. 3A and 3B), total inflammatory lesion count (sum of papules, pustules, and nodules) (Tables 4A, 4B, 4C, 4D, 4E, 4F, and FIGS. 4A and 4B), and Clinician's Erythema Assessment (CEA) (Tables 5A, 5B and FIGS. 5A and 5B), at Weeks 2, 4, 8, 12, and 16 compared to Baseline. The impact of the treatment on the quality of life (QoL) of the subjects was assessed using the rosacea-specific tool RosaQoL (Tables 6A, 6B, 6C, 6D) in addition to the Dermatology Life Quality Index (DLQI) (Tables 7A, 7B, 7C, 7D) at Baseline and Weeks 2, 4, 8, 12, and 16

Example 4. Treatment Success of Rosacea based on Investigator's Global Assessment modified scale without erythema (IGA) was evaluated at Weeks 2, 4, 8, 12, and 16 using 40 mg Minocycline HCl of Example 1 (DFD-29), 40 mg Doxycycline (Oracea), or Placebo. Treatment success is defined as IGA=0 or 1 and at least a 2-grade reduction from Baseline. The results of the two studies (MVOR-1 and MVOR-2) are shown in Tables 3A, 3B, 3C, 3D, and FIGS. 3A, 3B.

Compared to the Placebo group, the DFD-29 group had a significantly greater proportion of subjects with IGA treatment success at the Week 16 visit: 65,0% vs 31.2% (p<0.001) (MVOR-1) and 60.1% vs 26.8% (p<0.001) (MVOR-2). Compared to the Doxycycline group, the DFD-29 group had a significantly greater proportion of subjects with IGA ‘treatment success’ at the Week 16 visit: 65.0% vs 46.1% (p=0.014) (MVOR-1) and 60.1% vs 31.4% (p<0.001) (MVOR-2).

Notably, by Fitzpatrick skin type, IGA ‘treatment success’ with DFD-29, Doxycycline, and Placebo was achieved in 66%, 46%, and 32%, respectively, of subjects with type I, II, or III and in 59%, 55%, and 29%, respectively, of subjects with type IV, V, or VI. Accordingly, no significant difference was observed in efficacy of DFD-29 in IGA treatment success across Fitzpatrick skin types.

TABLE 3A
Subjects with IGA Treatment Success (Minocycline vs. Placebo, MVOR-1)

| | IGA Treatment | | | | | | | |
|---|---|---|---|---|---|---|---|---|
| Visit | Success¹ | DFD-29 | | Placebo | | Difference (95% CI) | | p-values |
| Week 2 | | (N = 121) | | (N = 76) | | | | |
| | n (%) | 15 | (12.4%) | 1 | (1.3%) | 10.5% | (4.46%, 16.51%) | 0.005² |
| Week 4 | | (N = 120) | | (N = 75) | | 21.5% | (11.79%, 31.18%) | <0.001² |
| | n (%) | 35 | (29.2%) | 6 | (8.0%) | | | |
| Week 8 | | (N = 119) | | (N = 75) | | 31.2% | (20.27%, 42.20%) | <0.001² |
| | n (%) | 58 | (48.7%) | 13 | (17.3%) | | | |
| Week 12 | | (N = 118) | | (N = 74) | | | | |
| | n (%) | 68 | (57.6%) | 13 | (17.6%) | 39.8% | (27.78%, 51.79%) | <0.001² |
| | IGA Treatment | | | | | | | |
| | Success¹ | DFD-29 | | Placebo | | Difference (95% CI) | | p-values |
| Week 16 | | 122 | | 80 | | | | |
| | n (%) | 79 | (65.0%)² | 25 | (31.2%)² | 32.9% | (19.62%, 46.19%) | <0.001³ |

^{2,3}p-values for treatment comparisons from Cochran-Mantel
Haenszel test for general association, adjusted for analysis center.

TABLE 3B
Subjects with IGA Treatment Success (Minocycline vs. Doxycycline, MVOR-1)

| | IGA Treatment | | | | | | | |
|---|---|---|---|---|---|---|---|---|
| Visit | Success¹ | DFD-29 | | Doxycycline | | Difference (95% CI) | | p-values |
| Week 2 | | (N = 121) | | (N = 115) | | | | |
| | n (%) | 15 | (12.4%) | 10 | (8.7%) | 3.7% | (−3.45%, 10.91%) | 0.318² |
| Week 4 | | (N = 120) | | (N = 114) | | | | |
| | n (%) | 35 | (29.2%) | 21 | (18.4%) | 9.7% | (−0.22%, 19.57%) | 0.054² |
| Week 8 | | (N = 119) | | (N = 107) | | | | |
| | n (%) | 58 | (48.7%) | 30 | (28.0%) | 20.0% | (8.88%, 31.15%) | <0.001² |
| Week 12 | | (N = 118) | | (N = 101) | | | | |
| | n (%) | 68 | (57.6%) | 43 | (42.6%) | 14.4% | (2.19%, 26.52%) | 0.019² |
| Week 16 | | (N = 122) | | (N = 121) | | | | |
| | n (%) | 79 | (65.0%)² | 56 | (46.1%)² | 18.0% | (4.98%, 31.05%) | 0.014³ |

^{2,3}p-values for treatment comparisons from Cochran-Mantel
Haenszel test for general association, adjusted for analysis center.

TABLE 3C
Subjects with IGA Treatment Success (Minocycline vs. Placebo, MVOR-2)

| | IGA Treatment | | | | | | | |
|---|---|---|---|---|---|---|---|---|
| Visit | Success¹ | DFD-29 | | Placebo | | Difference (95% CI) | | p-values |
| Week 2 | | (N = 121) | | (N = 80) | | | | |
| | n (%) | 13 | (10.7%) | 3 | (3.8%) | 7.0% | (0.32%, 13.60%) | 0.045² |
| Week 4 | | (N = 119) | | (N = 78) | | | | |
| | n (%) | 32 | (26.9%) | 7 | (9.0%) | 16.5% | (7.41%, 25.53%) | 0.001² |
| Week 8 | | (N = 117) | | (N = 74) | | | | |
| | n (%) | 45 | (38.5%) | 8 | (10.8%) | 26.6% | (15.82%, 37.36%) | <0.001² |
| Week 12 | | (N = 116) | | (N = 71) | | | | |
| | n (%) | 53 | (45.7%) | 9 | (12.7%) | 35.6% | (25.21%, 46.00%) | <0.001² |
| | IGA Treatment | | | | | | | |

| | Success¹ | DFD-29 | Placebo | Difference (95% CI) | p-values |
|---|---|---|---|---|---|
| Week 16 | n (%) | (N = 123) | (N = 82) | | |

| | | | | | | |
|---|---|---|---|---|---|---|
| 74 | (60.1%)² | 22 | (26.8%)² | 34.1% | (21.32%, 46.80%) | <0.001³ |

^{2,3}p-values for treatment comparisons from Cochran-Mantel
Haenszel test for general association, adjusted for analysis center.

TABLE 3D
Subjects with IGA Treatment Success (Minocycline vs. Doxycycline, MVOR-2)

| | IGA Treatment | | | | | | | |
|---|---|---|---|---|---|---|---|---|
| Visit | Success¹ | DFD-29 | | Doxycycline | | Difference (95% CI) | | p-values |
| Week 2 | | (N = 121) | | (N = 117) | | | | |
| | n (%) | 13 | (10.7%) | 7 | (6.0%) | 5.4% | (−0.86%, 11.59%) | 0.086² |
| Week 4 | | (N = 119) | | (N = 119) | | | | |
| | n (%) | 32 | (26.9%) | 10 | (8.4%) | 18.1% | (9.48%, 26.80%) | <0.001² |
| Week 8 | | (N = 117) | | (N = 120) | | | | |
| | n (%) | 45 | (38.5%) | 23 | (19.2%) | 18.7% | (8.38%, 29.04%) | <0.001² |
| Week 12 | | (N = 116) | | (N = 114) | | | | |
| | n (%) | 53 | (45.7%) | 26 | (22.8%) | 23.2% | (12.52%, 33.96%) | <0.001² |
| | IGA Treatment | | | | | | | |
| | Success¹ | DFD-29 | | Placebo | | Difference (95% CI) | | p-values |
| Week 16 | | (N = 123) | | (N = 125) | | | | |
| | n (%) | 74 | (60.1%)² | 39 | (31.4%)² | 28.3% | (17.39%, 39.31%) | <0.001³ |

^{2,3}p-values for treatment comparisons from Cochran-Mantel
Haenszel test for general association, adjusted for analysis center.

Example 5. Total Lesion Count. Minocycline HCl and 40 mg Doxycycline of Example 1, or Placebo. The total lesion count present at the end of the study is presented relative to total number present at baseline (Day 1) of study. Results are shown in Tables 4A, 4B, 4C, 4D, 4E, 4F, and FIGS. 4A, 4B.

Compared to the Placebo group, the DFD-29 group had a significantly greater reduction in total inflammatory lesion count from Baseline to the Week 16 visit: −21.3 vs −12.1 (p<0.001) (MVOR-1) and −18.0 (0.66) vs −11.1 (0.86) (p<0.001) (MVOR-2). Compared to the Doxycycline group, the DFD-29 group had a significantly greater reduction in total inflammatory lesion count from Baseline to the Week 16 visit, −20.5 vs −15.8 (p<0.001) (MVOR-1) and −18.4 (0.71) vs −14.9 (0.71) (p<0.001) (MVOR-2).

Notably, Mean (SD) change from Baseline in total inflammatory lesion count at Week 16 with DFD-29, Doxycycline, and Placebo was-21.6 (11.18), −15.1 (9.64), and −12.0 (9.98), respectively, for subjects with Fitzpatrick skin type I, II, or III, and −20.0 (11.65), −14.2 (7.54), and −12.3 (5.62), respectively, for subjects with type IV, V, or VI. Accordingly, no significant difference was observed in efficacy of DFD-29 in total inflammatory lesion count across Fitzpatrick skin types.

TABLE 4A
Total Inflammatory Lesion Count (Minocycline vs. Placebo, MVOR-1)

| | | DED-29 | Placebo | | | | |
|---|---|---|---|---|---|---|---|
| Visit | Parameter | LS Mean (SE) | LS Mean (SE) | | Difference (SE) (95% CI) | | p-values |
| Overall | Treatment | −17.1 (0.62)¹ | −8.9 | (0.79)¹ | −8.2 (0.96) | [−10.09, −6.30]¹ | <0.001¹ |
| | Analysis center | | | | | | <0.001¹ |
| | Visit | | | | | | <0.001¹ |
| | Treatment*Visit | | | | | | <0.001¹ |
| | Baseline | | | | | | <0.001¹ |
| Week 2 | Treatment | −11.4 (0.71)¹ | −6.9 | (0.89)¹ | −4.6 (1.09) | [−6.72, −2.40]¹ | <0.001² |
| Week 4 | Treatment | −16.7 (0.74)¹ | −7.7 | (0.94)¹ | −9.0 (1.16) | [−11.27, −6.70]¹ | <0.001² |
| Week 8 | Treatment | −19.3 (0.78)¹ | −10.5 | (0.98)¹ | −8.8 (1.21) | [−11.20, −6.41]¹ | <0.001² |
| Week 12 | Treatment | −20.8 (0.75)¹ | −10.3 | (0.95)¹ | −10.4 (1.17) | [−12.74, −8.12]¹ | <0.001² |
| | | DFD-29 | Placebo | | | | |
| | Parameter | LS Mean (SE) | LS Mean (SE) | | Difference (SE) (95% CI) | | p-values |
| Week 16 | | (N = 122) | (N = 80) | | | | |
| | Treatment | −21.3 (0.77)¹ | −12.1 | (0.97)¹ | −9.2 (1.18) | [−11.49, −6.87]¹ | <0.001¹ |
| | Analysis center | | | | | | 0.050¹ |
| | Baseline | | | | | | <0.001¹ |

Notes:
LS Mean = least squares mean, SE = standard error, Overall is Weeks 2-16.
¹p-values from mixed model repeated measures i^{th} treatment, analysis center, visit, and treatment by visit interaction as fixed factors, Baseline total inflammatory lesion count as a covariate, and subject as a random factor.
²p-values for treatment comparisons using contrasts.

TABLE 4B
Total Inflammatory Lesion Count (Minocycline vs. Doxycycline, MVOR-1)

| | | DFD-29 | Doxycycline | | | |
|---|---|---|---|---|---|---|
| Visit | Parameter | LS Mean (SE) | LS Mean (SE) | | Difference (SE) (95% CI) | p-values |
| Overall | Treatment | −16.7 (0.58)¹ | −12.2 | (0.59)¹ | −4.5 (0.81) [−6.06, −2.87]¹ | <0.001¹ |
| | Analysis center | | | | | <0.001¹ |
| | Visit | | | | | <0.001¹ |
| | Treatment*Visit | | | | | 0.006¹ |
| | Baseline | | | | | <0.001¹ |
| Week 2 | Treatment | −11.1 (0.67)¹ | −8.9 | (0.68)¹ | −2.2 (0.93) [−4.03, −0.35]¹ | 0.020² |
| Week 4 | Treatment | −16.4 (0.70)¹ | −11.1 | (0.71)¹ | −5.3 (0.98) [−7.21, −3.33]¹ | <0.001² |
| Week 8 | Treatment | −19.0 (0.73)¹ | −13.3 | (0.76)¹ | −5.6 (1.04) [−7.68, −3.58]¹ | <0.001² |
| Week 12 | Treatment | −20.4 (0.69)¹ | −15.7 | (0.73)¹ | −4.8 (0.99) [−6.71, −2.80]¹ | <0.001² |
| | | DFD-29 | Doxycycline | | | |
| | Parameter | LS Mean (SE) | LS Mean (SE) | | Difference (SE) (95% CI) | p-values |
| Week 16 | | (N = 122) | (N = 121) | | | |
| | Treatment | −20.5 (0.69)¹ | −15.8 | (0.73)¹ | −4.7 (0.98) [−6.67, −2.83]¹ | <0.001¹ |
| | Analysis center | | | | | <0.001¹ |
| | Baseline | | | | | <0.001¹ |

Notes:
LS Mean = least squares mean, SE = standard error, Overall is Weeks 2-16.
¹p-values from mixed model repeated measures i^{th} treatment, analysis center, visit, and treatment by visit interaction as fixed factors, Baseline total inflammatory lesion count as a covariate, and subject as a random factor.
²p-values for treatment comparisons using contrasts.

TABLE 4C
Total Inflammatory Lesion Count (Minocycline vs. Placebo, MVOR-2)

| | | DFD-29 | | Placebo | | | | |
|---|---|---|---|---|---|---|---|---|
| Visit | Parameter | LS Mean (SE) | | LS Mean (SE) | | Difference (SE) (95% CI) | | p-values |
| Overall | Treatment | −13.4 | (0.51)¹ | −8.9 | (0.61)¹ | −4.5 (0.81) | [−6.06, −2.87]¹ | <0.001¹ |
| | Analysis center | | | | | | | <0.001¹ |
| | Visit | | | | | | | <0.001¹ |
| | Treatment*Visit | | | | | | | 0.042¹ |
| | Baseline | | | | | | | <0.001¹ |
| Week 2 | Treatment | −912 | (0.54)¹ | −6.4 | (0.68)¹ | −2.8 (0.81) | [−4.41, −1.21]¹ | <0.001² |
| Week 4 | Treatment | −12.5 | (0.67)¹ | −8.1 | (0.81)¹ | −4.4 (1.03) | [−6.42, −2.34]¹ | <0.001² |
| Week 8 | Treatment | −15.3 | (0.63)¹ | −10.1 | (0.76)¹ | −5.2 (0.96) | [−7.118, −3.32]¹ | <0.001² |
| Week 12 | Treatment | −16.7 | (0.64)¹ | −11.2 | (0.79)¹ | −5.6 (0.99) | [−7.55, −3.64]¹ | <0.001² |
| | | DFD-29 | | Placebo | | | | |
| | Parameter | LS Mean (SE) | | LS Mean (SE) | | Difference (SE) (95% CI) | | p-values |
| Week 16 | | (N = 123) | | (N = 82) | | | | |
| | Treatment | −18.0 | (0.66)¹ | −11.1 | (0.86)¹ | −4.8 (1.3) | [−8.87, −4.82]¹ | <0.001¹ |
| | Analysis center | | | | | | | <0.001¹ |
| | Baseline | | | | | | | <0.001¹ |

Notes:
LS Mean = least squares mean, SE = standard error, Overall is Weeks 2-16.
¹p-values from mixed model repeated measures i^{th} treatment, analysis center, visit, and treatment by visit interaction as fixed factors, Baseline total inflammatory lesion count as a covariate, and subject as a random factor.
²p-values for treatment comparisons using contrasts.

TABLE 4D
Total Inflammatory Lesion Count (Minocycline vs. Doxycycline, MVOR-2)

| | | DFD-29 | | Doxycycline | | | | |
|---|---|---|---|---|---|---|---|---|
| Visit | Parameter | LS Mean (SE) | | LS Mean (SE) | | Difference (SE) (95% CI) | | p-values |
| Overall | Treatment | −13.6 | (0.52)¹ | −8.9 | (0.79)¹ | −8.2 (0.96) | [−10.09, −6.30]¹ | 0.003¹ |
| | Analysis center | | | | | | | <0.001¹ |
| | Visit | | | | | | | <0.001¹ |
| | Treatment*Visit | | | | | | | 0.812¹ |
| | Baseline | | | | | | | <0.001¹ |
| Week 2 | Treatment | −9.4 | (0.55)¹ | −7.4 | (0.56)¹ | −2.0 (0.75) | [−3.52, −0.54]¹ | 0.008² |
| Week 4 | Treatment | −12.6 | (0.61)¹ | −10.2 | (0.61)¹ | −2.5 (0.84) | [−4.13, −0.84]¹ | 0.003² |
| Week 8 | Treatment | −15.5 | (0.59)¹ | −13.4 | (0.58)¹ | −2.1 (0.80) | [−3.69, −0.52]¹ | 0.009² |
| Week 12 | Treatment | −16.9 | (0.70)¹ | −15.0 | (0.70)¹ | −1.9 (0.96) | [−3.79, −0.02]¹ | 0.052² |
| | | DFD-29 | | Doxycycline | | | | |
| | Parameter | LS Mean (SE) | | LS Mean (SE) | | Difference (SE) (95% CI) | | p-values |
| Week 16 | | (N = 123) | | (N = 125) | | | | |
| | Treatment | −18.4 | (0.71)¹ | −14.9 | (0.71)¹ | −3.5 (0.96) | [−5.35, −1.59]¹ | <0.001¹ |
| | Analysis center | | | | | | | <0.001¹ |
| | Baseline | | | | | | | <0.001¹ |

Notes:
LS Mean = least squares mean, SE = standard error, Overall is Weeks 2-16.
¹p-values from mixed model repeated measures i^{th} treatment, analysis center, visit, and treatment by visit interaction as fixed factors, Baseline total inflammatory lesion count as a covariate, and subject as a random factor.
²p-values for treatment comparisons using contrasts.

TABLE 4E
Total Inflammatory Lesion Count-Percent Change from Baseline, MVOR-1.

| Visit | Actual Value | | | | | | Percentage Change from Baseline | | | | | |
|---|---|---|---|---|---|---|---|---|---|---|---|---|
| Treatment | N | Mean | SD | Median | Min | Max | N | Mean | SD | Median | Min | Max |
| Baseline | | | | | | | | | | | | |
| DFD-29 | 121 | 26.4 | 10.50 | 24.0 | 15 | 58 | | | | | | |
| Doxycycline | 116 | 23.8 | 8.88 | 22.0 | 15 | 56 | | | | | | |
| Placebo | 77 | 26.2 | 8.89 | 23.0 | 15 | 58 | | | | | | |
| Week 2 | | | | | | | | | | | | |
| DFD-29 | 121 | 15.5 | 10.49 | 14.0 | 0 | 60 | 121 | −40.2 | 33.24 | −37.5 | −100 | 150 |
| Doxycycline | 114 | 16.5 | 10.13 | 16.0 | 0 | 67 | 114 | −31.5 | 31.51 | −26.2 | −100 | 47 |
| Placebo | 75 | 20.1 | 10.91 | 19.0 | 1 | 81 | 75 | −24.0 | 24.70 | −21.1 | −94 | 40 |
| Week 4 | | | | | | | | | | | | |
| DFD-29 | 120 | 10.2 | 7.86 | 8.5 | 0 | 37 | 120 | −59.2 | 30.31 | −60.6 | −100 | 88 |
| Doxycycline | 114 | 14.1 | 11.05 | 12.0 | 0 | 70 | 114 | −40.9 | 37.96 | −38.5 | −100 | 124 |
| Placebo | 74 | 18.9 | 12.07 | 16.5 | 0 | 54 | 74 | −29.9 | 33.45 | −29.3 | −100 | 52 |
| Week 8 | | | | | | | | | | | | |
| DFD-29 | 119 | 7.6 | 7.54 | 5.0 | 0 | 35 | 119 | −69.7 | 29.36 | −77.3 | −100 | 56 |
| Doxycycline | 107 | 11.7 | 10.28 | 10.0 | 0 | 59 | 107 | −52.0 | 36.21 | −55.0 | −100 | 95 |
| Placebo | 74 | 16.0 | 13.02 | 14.0 | 0 | 76 | 74 | −42.5 | 32.08 | −41.9 | −100 | 38 |
| Week 12 | | | | | | | | | | | | |
| DFD-29 | 118 | 6.2 | 6.57 | 4.0 | 0 | 33 | 118 | −75.0 | 27.13 | −81.9 | −100 | 75 |
| Doxycycline | 101 | 8.9 | 8.13 | 8.0 | 0 | 42 | 101 | −61.8 | 31.90 | −67.6 | −100 | 50 |
| Placebo | 73 | 16.1 | 11.44 | 15.0 | 0 | 66 | 73 | −40.4 | 30.82 | −39.1 | −100 | 27 |
| Week 16 | | | | | | | | | | | | |
| DFD-29 | 117 | 5.2 | 6.04 | 3.0 | 0 | 30 | 117 | −79.0 | 23.22 | −86.4 | −100 | 5 |
| Doxycycline | 98 | 8.4 | 8.89 | 7.0 | 0 | 40 | 98 | −64.8 | 32.59 | −67.6 | −100 | 62 |
| Placebo | 73 | 13.9 | 11.68 | 11.0 | 0 | 60 | 73 | −48.2 | 35.88 | −51.6 | −100 | 95 |

Notes:
SD = standard deviation.

TABLE 4F
Total Inflammatory Lesion Count-Percent Change from Baseline, MVOR-2.

| Visit | Actual Value | | | | | | Percentage Change from Baseline | | | | | |
|---|---|---|---|---|---|---|---|---|---|---|---|---|
| Treatment | N | Mean | SD | Median | Min | Max | N | Mean | SD | Median | Min | Max |
| Baseline | | | | | | | | | | | | |
| DFD-29 | 122 | 23.9 | 7.50 | 22.0 | 15 | 49 | | | | | | |
| Doxycycline | 121 | 25.2 | 9.16 | 23.0 | 15 | 55 | | | | | | |
| Placebo | 82 | 24.2 | 7.87 | 22.0 | 15 | 57 | | | | | | |
| Week 2 | | | | | | | | | | | | |
| DFD-29 | 121 | 15.9 | 8.99 | 15.0 | 2 | 41 | 121 | −33.5 | 29.57 | −24.4 | −95 | 13 |
| Doxycycline | 117 | 18.8 | 9.65 | 18.0 | 0 | 56 | 117 | −26.4 | 25.05 | −23.5 | −100 | 32 |
| Placebo | 80 | 18.6 | 8.01 | 19.0 | 1 | 37 | 80 | −23.4 | 27.30 | −20.3 | −93 | 44 |
| Week 4 | | | | | | | | | | | | |
| DFD-29 | 119 | 12.7 | 9.52 | 11.0 | 0 | 40 | 119 | −48.1 | 33.25 | −47.6 | −100 | 31 |
| Doxycycline | 119 | 16.0 | 9.25 | 15.0 | 2 | 58 | 119 | −36.9 | 28.38 | −37.5 | −95 | 88 |
| Placebo | 78 | 17.1 | 10.33 | 14.0 | 2 | 64 | 78 | −30.0 | 37.06 | −37.2 | −59 | 113 |
| Week 8 | | | | | | | | | | | | |
| DFD-29 | 117 | 9.7 | 8.01 | 8.0 | 0 | 29 | 117 | −60.1 | 30.27 | −61.8 | −100 | 10 |
| Doxycycline | 120 | 12.8 | 8.63 | 12.0 | 0 | 52 | 120 | −49.6 | 27.37 | −50.9 | −100 | 38 |
| Placebo | 74 | 15.1 | 9.56 | 14.0 | 0 | 52 | 74 | −37.6 | 39.80 | −47.0 | −100 | 111 |
| Week 12 | | | | | | | | | | | | |
| DFD-29 | 115 | 8.3 | 7.74 | 6.0 | 0 | 32 | 116 | −66.0 | 29.54 | −71.5 | −100 | 6 |
| Doxycycline | 114 | 10.8 | 8.02 | 10.0 | 0 | 45 | 114 | −55.3 | 32.99 | −58.7 | −100 | 114 |
| Placebo | 71 | 14.2 | 9.67 | 12.0 | 0 | 37 | 71 | −42.3 | 38.47 | −52.2 | −100 | 56 |
| Week 16 | | | | | | | | | | | | |
| DFD-29 | 115 | 6.5 | 7.38 | 3.0 | 0 | 30 | 415 | −74.2 | 28.56 | −85.7 | −100 | 15 |
| Doxycycline | 113 | 10.3 | 9.41 | 8.0 | 0 | 57 | 113 | −58.6 | 35.29 | −63.0 | −100 | 110 |
| Placebo | 68 | 13.1 | 10.91 | 10.5 | 0 | 42 | 58 | −47.7 | 41.26 | −55.4 | −100 | 50 |

Notes:
SD = standard deviation.

Example 6. Clinician's Erythema Assessment (CEA). The CEA was carried out by visual inspection by the Investigator or delegated Sub-Investigator at every study visit from Screening at Weeks 2, 4, 8, 12, and 16. As far as was possible, the same study staff member was to perform the assessment on an individual subject at all visits. The erythema assessment was carried out separately at 5 locations on the face: forehead, nose, chin, right cheek, and left cheek. A ≥2-grade reduction from Baseline to Week 16 was assessed on the most severe CEA score/s out of the 5 locations on the face. If more than one location had the most severe scores, then the ≥2-grade reduction had to be on the average scores for those locations at Week 16 (e.g., if the Baseline CEA scores for the right and left cheeks were 3, the nose was 1, and the forehead and chin were 0 each, the Baseline average score was equal to 3 for the right and left cheeks, and the average of the scores for the two cheeks at Week 16 had to be 1 or below to be considered as at least a 2-grade reduction on the CEA). Subjects with a >2-grade reduction in the worst affected area/s who showed worsening (increases) by >1 grade over Baseline in one or more of the other facial locations were considered treatment non-responders for the primary analysis of this endpoint and considered treatment responders for the supportive analyses. DFD-29 was statistically superior to placebo in reducing the clinician's erythema assessment score by at least 2 grades from baseline (CEA Success) at 16 weeks. Results are shown in Tables 5A, 5B, 5C, 5D, and FIGS. 5A, 5B.

Compared to the Placebo group, the DFD-29 group had a significantly greater proportion of subjects with at least a 2-grade reduction in CEA score from Baseline to Week 16, 31.7% vs 13.8% (p=0.006) (MVOR-1) and 24.5% vs 12.0% (p=0.023) (MVOR-2).

TABLE 5A
CEA for DFD-29 Versus Placebo, MVOR-1

| | Two-Grade | | | | | | | |
|---|---|---|---|---|---|---|---|---|
| | Reduction¹ | DFD-29 | | Placebo | | Difference (95% CI) | | p-values |
| Week 2 | | (N = 121) | | (N = 76) | | | | |
| | n (%) | 12 | (9.9%) | 1 | (1.3%) | 8.5% | (2.64%, 14.45%) | 0.019² |
| Week 4 | | (N = 120) | | (N = 75) | | | | |
| | n (%) | 17 | (14.2%) | 0 | | 13.5% | (7.33%, 19.75%) | 0.001² |
| Week 8 | | (N = 119) | | (N = 75) | | | | |
| | n (%) | 29 | (24.4%) | 6 | (8.0%) | 15.9% | (6.77%, 25.08%) | 0.002² |
| Week 12 | | (141 = 118) | | (N = 74) | | | | |
| | n (%) | 28 | (23.7%) | 6 | (8.1%) | 15.0% | (6.15%, 23.92%) | 0.004² |
| | 2-Grade | | | | | | | |
| | Reduction¹ | DFD-29 | | Placebo | | Difference (95% CI) | | p-values |
| Week 16 | | (N = 122) | | (N = 80) | | | | |
| | n (%) | 39 | (31.7%)² | 11 | (13.8%)² | 18.1% | (7.32%, 28.96%) | 0.006³ |

¹2-grade improvement is defined as a >=2-grade reduction in the same location(s) as the Baseline score. If more than one location was used for Baseline, the same locations will be averaged for the post-baseline score.
²p-values for treatment comparisons from Cochran-Mantel-Haenszel test for general association, adjusted for analysis center.
²Frequency and Proportion derived as the average of the frequencies and proportions of subjects with at least 2-grade improvement from the 200 imputed datasets.
³p-values for treatment comparisons from Cochran-Mantel-Haenszel test for general association, adjusted for analysis center and derived via Proc MIANALYZE from the analysis results performed on the 200 imputed datasets.

TABLE 5B
CEA for DFD-29 Versus Doxycycline, MVOR-1

| | Two-Grade | | | | | | | |
|---|---|---|---|---|---|---|---|---|
| Visit | Reduction)¹ | DFD-29 | | Doxycycline | | Difference (95% CI) | | p-values |
| Week 2 | | (N = 121) | | (N = 115) | | | | |
| | n (%) | 12 | (9.9%) | 4 | (3.5%) | 6.3% | (0.02%, 12.64%) | 0.052² |
| Week 4 | | (N = 120) | | (N = 114) | | | | |
| | n (%) | 17 | (14.2%) | 9 | (7.9%) | 5.4% | (−2.27%, 13.03%) | 0.174² |
| Week 8 | | (N = 119) | | (N = 107) | | | | |
| | n(%) | 29 | (24.4%) | 13 | (12.1%) | 11.1% | (1.38%, 20.81%) | 0.024² |
| Week 12 | | (N = 118) | | (N = 101) | | | | |
| | n (%) | 28 | (23.7%) | 20 | (19.8%) | 4.3% | (−6.18%, 14.82%) | 0.419² |
| Week 16 | | (N = 117) | | (N = 98) | | | | |
| | n(%) | 38 | (32.5%) | 26 | (26.5%) | 6.8% | (−4.40%, 17.95%) | 0.239² |

For each patient, baseline CEA scores include the facial location(s) with the worst (highest) scores. Post-baseline CEA scores include the average of the scores from the same location(s) that were identified as worst at Baseline.
¹Includes subjects with any 2-grade reduction.
²p-values for treatment comparisons from Cochran-Mantel-Haenszel test for general association, adjusted for analysis center.

TABLE 5C
CEA for DFD-29 Versus Placebo, MVOR-2

| | Two-Grade | | | | | | | |
|---|---|---|---|---|---|---|---|---|
| Visit | Reduction)¹ | DFD-29 | | Placebo | | Difference (95% CI) | | p-values |
| Week 2 | | (N = 121) | | (N = 80) | | 2.5% | (−4.10%, 9.09%) | 0.490² |
| | n (%) | 9 | (7.4%) | 4 | (5.0%) | | | |
| Week 4 | | (N = 119) | | (N = 78) | | 6.0% | (−3.50%, 15.54%) | 0.243² |
| | n (%) | 20 | (16.8%) | 10 | (12.8%) | | | |
| Week 8 | | (N = 117) | | (N = 74) | | 13.0% | (3.01%, 22.94%) | 0.017² |
| | n (%) | 25 | (21.4%) | 9 | (12.2%) | | | |
| Week 12 | | (N = 116) | | (N = 71) | | 15.3% | (5.01%, 25.52%) | 0.006² |
| | n (%) | 27 | (23.3%) | 9 | (12.7%) | | | |
| | 2-Grade | | | | | | | |
| | Reduction¹ | DFD-29 | | Placebo | | Difference (95% CI) | | p-values |
| Week 16 | | (N = 123) | | (N = 82) | | | | |
| | n (%) | 30 | (24.5%) | 10 | (12.0%) | 13.9% | (3.71%, 23.99%) | 0.23³ |

TABLE 5D
CEA for DFD-29 Versus Doxycycline, MVOR-2

| | Two-Grade | | | | | | | |
|---|---|---|---|---|---|---|---|---|
| Visit | Reduction¹ | DFD-29 | | Doxycycline | | Difference (95% CI) | | p-values |
| Week 2 | | (N = 121) | | (N = 117) | | | | |
| | n (%) | 9 | (7.4%) | 5 | (4.3%) | 3.1% | (−2.72%, 8.87%) | 0.316² |
| Week 4 | | (N = 119) | | (N = 119) | | | | |
| | n (%) | 20 | (16.8%) | 3 | (6.7%) | 9.7% | (1.71%, 17.70%) | 0.020² |
| Week 8 | | (N = 117) | | (N = 120) | | | | |
| | n (%) | 25 | (21.4%) | 22 | (18.3%) | 2.7% | (−7.39%, 12.80%) | 0.600² |
| Week 12 | | (N = 116) | | (N = 114) | | | | |
| | n (%) | 27 | (23.3%) | 19 | (16.7%) | 6.4% | (−3.45%, 16.27%) | 0.200² |
| Week 16 | | (N = 114) | | (N = 113) | | | | |
| | n (%) | 28 | (24.6%) | 24 | (21.2%) | 3.3% | (−7.16%, 13.68%) | 0.540² |

¹2-grade improvement is defined as a >=2-grade reduction in the same location(s) as the Baseline score. If more than one location was used for Baseline, the same locations will be averaged for the post-baseline score.
²p-values for treatment comparisons from Cochran-Mantel-Haenszel test for general association, adjusted for analysis center.

Example 7. Rosacea Quality of Life. Rosacea Quality of Life (RosaQoL.) The RosaQoL assessment was carried out by asking questions as per the validated RosaQoL questionnaire instrument at every study visit from Baseline through Week 16. Subjects rated 21 questions on a 5-grade scale and their perception of the impact that rosacea had on various dimensions influencing their quality of life,

TABLE 6A
Rosacea Quality of Life, DFD-29 Versus Placebo, MVOR-1

| | | DFD-29 | | Placebo | | | |
|---|---|---|---|---|---|---|---|
| Visit | Parameter | LS Mean (SE) | | LS Mean (SE) | Difference (SE) (95% CI) | | p-values |
| Overall | Treatment | −12.0 | (0.91)¹ | −5.7 (1.15)¹ | −6.3 (1.42) | [−9.14, −3.54]¹ | <0.001¹ |
| | Analysis center | | | | | | 0.130¹ |
| | Visit | | | | | | <0.001¹ |
| | Treatment*Visit | | | | | | <0.001¹ |
| | Baseline | | | | | | 0.001¹ |
| Week 2 | Treatment | −4.9 | (0.80)¹ | −3.5 (1.01)¹ | −1.4 (1.23) | [−3.87, 0.98]¹ | 0.241² |
| Week 4 | Treatment | −9.6 | (0.89)¹ | −4.7 (1.11)¹ | 4.9 (1.38) | [−7.63, −2.20]¹ | <0.001² |
| Week 8 | Treatment | −12.5 | (1.15)¹ | −7.2 (1.45)¹ | −5.3 (1.82) | [−8.87, −1.70]¹ | 0.004² |
| Week 12 | Treatment | −15.6 | (1.27)¹ | −7.1 (1.60)¹ | −8.5 (2.01) | [−12.43, −4.49]¹ | <0.001² |
| Week 16 | Treatment | −17.6 | (1.36)¹ | −6.0 (1.71)¹ | −11.6 (2.15) | [15.84, −7.34]¹ | <0.001² |

Notes:
LS Mean = least squares mean, SE = standard error, Overall is Weeks 2-16.
¹p-values from mixed model repeated measures with treatment, analysis center, visit, and treatment by visit interaction as fixed factors, Baseline Rosacea Quality of Life Score as a covariate, and subject as a random factor.
²p-values for treatment comparisons using contrasts.

TABLE 6B
Rosacea Quality of Life, DFD-29 Versus Doxycycline, MVOR-1

| | | DFD-29 | | Doxycycline | | | | |
|---|---|---|---|---|---|---|---|---|
| Visit | Parameter | LS Mean (SE) | | LS Mean (SE) | | Difference (SE)195% CI) | | p-values |
| Overall | Treatment | −11.8 | (1.05)¹ | −9.8 | (1.08)¹ | −2.0 (1.46) | [−4.90, 0.85]¹ | 0.166¹ |
| | Analysis center | | | | | | | 0.935¹ |
| | Visit | | | | | | | <0.001¹ |
| | Treatment*Visit | | | | | | | 0.133¹ |
| | Baseline | | | | | | | <0.001¹ |
| Week 2 | Treatment | −4.7 | (1.02)¹ | −5.1 | (1.04)¹ | 0.4 (1.41) | [−2.36, 3.21]¹ | 0.766² |
| Week 4 | Treatment | −9.3 | (1.12)¹ | −7.4 | (1.14)¹ | −1.9 (1.56) | (−4.98, 1.17]¹ | 0.223² |
| Week 8 | Treatment | −12.3 | (1.15)¹ | −10.1 | (1.19)¹ | −2.2 (1.61) | [−5.33, 1.02]¹ | 0.183² |
| Week 12 | Treatment | −15.3 | (1.27)¹ | −11.8 | (1.33)¹ | −33 (1.80) | [−7.08, 0.03]¹ | 0.052² |
| Week 16 | Treatment | −17.3 | (1.43)¹ | −14.3 | (1.51)¹ | −3.0 (2.05) | [−7.01, 1.06]¹ | 0.148² |

Notes:
LS Mean = least squares mean, SE = standard error, Overall is Weeks 2-16.
¹p-values from mixed model repeated measures with treatment, analysis center, visit, and treatment by visit interaction as fixed factors, Baseline Rosacea Quality of Life Score as a covariate, and subject as a random factor.
²p-values for treatment comparisons using contrasts.

TABLE 6C
Rosacea Quality of Life, DFD-29 Versus Placebo, MVOR-2

| | | DFD-29 | | Placebo | | | |
|---|---|---|---|---|---|---|---|
| Visit | Parameter | LS Mean (SE) | | LS Mean (SE) | Difference (SE) [95% CI′ | | p-values |
| Overall | Treatment | −12.7 | (0.92)¹ | −6.0 (1.11)¹ | −6.7 (1.39) | (−9.46, −3.97]¹ | <0.001¹ |
| | Analysis center | | | | | | 0.258¹ |
| | Visit | | | | | | <0.00¹ |
| | Treatment*Visit | | | | | | 0.002¹ |
| | Baseline | | | | | | 0.213¹ |
| Week 2 | Treatment | −6.7 | (0.88)¹ | −4.0 (1.05)¹ | −2.7 (1.31) | [−5.24, −0.07]¹ | 0.044² |
| Week 4 | Treatment | −10.9 | (1.02)¹ | −5.5 (1.23)¹ | −5.3 (1.55) | [−8.39, −2.29]¹ | <0.001² |
| Week 8 | Treatment | −13.9 | (1.04)¹ | −5.8 (1.26)¹ | −8.1 (1.59) | [−11.22, −4.94]¹ | <0.001² |
| Week 12 | Treatment | −15.9 | (1.17)¹ | −7.6 (1.45)¹ | −8.3 (1.83) | [−11.93, −4.72]¹ | <0.001² |
| Week 16 | Treatment | −16.2 | (1.24)¹ | −7.1 (1.54)¹ | −9.2 (1.94) | [−12.99, −5.35]¹ | <0.001² |

Notes:
LS Mean = least squares mean, SE = standard error.
¹p-values from mixed model repeated measures with treatment, analysis center, visit, and treatment by visit interaction as fixed factors, Baseline Rosacea Quality of Life Score as a covariate, and subject as a random factor.
²p-values for treatment comparisons using contrasts.

TABLE 6D
Rosacea Quality of Life, DFD-29 Versus Doxycycline, MVOR-2

| | | DFD-29 | | Doxycydine | | | | |
|---|---|---|---|---|---|---|---|---|
| Visit | Parameter | LS Mean (SE) | | LS Mean (SE) | | Difference (SE) [95% CI) | | p-values |
| Overall | Treatment | −13.0 | (0.96)¹ | −10.4 | (0.96)¹ | −2.6 (1.30) | [−5.14, −0.01]¹ | 0.049¹ |
| | Analysis center | | | | | | | 0.541¹ |
| | Visit | | | | | | | <0.001¹ |
| | Treatment*Visit | | | | | | | 0.693¹ |
| | Baseline | | | | | | | 0.003¹ |
| Week 2 | Treatment | −7.0 | (0.92)¹ | −5.2 | (0.93)¹ | −1.7 (1.25) | [−4.19, 0.72]¹ | 0.166² |
| Week 4 | Treatment | −11.1 | (1.05)¹ | −8.9 | (1.05)¹ | −2.2 (1.44) | [−5.03, 0.63]¹ | 0.128² |
| Week 8 | Treatment | −14.1 | (1.11)¹ | −11.3 | (1.10)¹ | −2.8 (1.52) | [−5.83, 0.15]¹ | 0.063² |
| Week 12 | Treatment | −16.1 | (1.22)¹ | −12.5 | (1.22)¹ | −3.6 (1.69) | [−6.92, −0.27]¹ | 0.034² |
| Week 16 | Treatment | −16.5 | (1.25)¹ | −14.0 | (1.25)¹ | −2.5 (1.73) | [−5.93, 0.88]¹ | 0.145² |

Notes:
LS Mean = least squares mean, SE = standard error.
¹p-values from mixed model repeated measures with treatment, analysis center, visit, and treatment by visit interaction as fixed factors, Baseline Rosacea Quality of Life Score as a covariate, and subject as a random factor.
²p-values for treatment comparisons using contrasts.

Example 8. Dermatology Life Quality Index (DLQI). The DLQI questionnaire was carried out at every study visit from Baseline through Week 16 (or termination). The DLQI questionnaire consists of 10 questions covering the following topics: symptoms, embarrassment, shopping and home care, clothes, social and leisure, sport, work or study, close relationships, sexuality and treatment. Each question refers to the impact of the skin disease on the subject's life. Each question is scored from 0 to 3 and totaled, giving a possible score range from 0 (meaning no impact of skin disease on QoL) to 30 (meaning maximum impact on QoL).

TABLE 7A
Dermatology Life Quality Index, DFD-29 Versus Placebo, MVOR-1

| | | DFD-29 | Placebo | | | | |
|---|---|---|---|---|---|---|---|
| Visit | Parameter | IS Mean (SE) | IS Mean (SE) | | Difference (SE) [95% CI] | | p-values |
| Overall | Treatment | −3.6 (0.22)¹ | −23 | (0.28)¹ | −1.3 (0.34) | [−1.96, −0.62]¹ | <0.001¹ |
| | Analysis center | | | | | | 0.769¹ |
| | Visit | | | | | | <0.00¹ |
| | Treatment*Visit | | | | | | 0.035¹ |
| | Baseline | | | | | | <0.001¹ |
| Week 2 | Treatment | −2.6 (0.27)¹ | −1.6 | (0.34)¹ | −1.0 (0.43) | [−1.83, −0.15]¹ | 0.021² |
| Week 4 | Treatment | −3.1 (0.27)¹ | −2.5 | (0.34)¹ | −0.7 (0.43) | [−1.51, 0.17]¹ | 0.120² |
| Week 8 | Treatment | −4.0 (0.28)¹ | −23 | (0.35)¹ | −1.6 (0.44) | [−2.47, −0.74]¹ | <0.001² |
| Week 12 | Treatment | −4.3 (0.27)¹ | −2.4 | (0.34)¹ | −1.9 (0.42) | [−2.75, −1.10]¹ | <0.001² |
| Week 16 | Treatment | −4.1 (0.31)¹ | −2.8 | (0.39)¹ | −13 (0.48) | [−2.22, −0.32]¹ | 0.009² |

Notes:
LS Mean = least squares mean, SE = standard error.
¹p-values from mixed model repeated measures with treatment, analysis center, visit, and treatment by visit interaction as fixed factors, Baseline Dermatology Life Quality Index Score as a covariate, and subject as a random factor.
²p-values for treatment comparisons using contrasts.

TABLE 7B
Dermatology Life Quality Index, DFD-29 Versus Doxycycline, MVOR-1

| | | DFD-29 | Doxycycline | | | |
|---|---|---|---|---|---|---|
| Visit | Parameter | LS Mean (SE) | LS Mean (SE) | Difference (SE)195% CI) | | p-values |
| Overall | Treatment | −3.4 (0.21)¹ | −2.6 (0.22)¹ | −0.8 (0.29) | [−1.36, −0.20]¹ | 0.008¹ |
| | Analysis center | | | | | 0.758¹ |
| | Visit | | | | | <0.001¹ |
| | Treatment*Visit | | | | | 0.515¹ |
| | Baseline | | | | | <0.00¹ |
| Week 2 | Treatment | −2.4 (0.27)¹ | −1.8 (0.28)¹ | −0.6 (0.39) | [−1.34, 0.18]¹ | 0.134² |
| Week 4 | Treatment | −2.9 (0.28)¹ | −2.1 (0.29)¹ | −0.8 (0.39) | [−1.60, −0.05]¹ | 0.036² |
| Week 8 | Treatment | −3.8 (0.25)¹ | −2.6 (0.26)¹ | −1.1 (0.35) | [−1.80, −0.41]¹ | 0.002² |
| Week 12 | Treatment | −4.1 (0.24)¹ | −3.3 (0.25)¹ | −0.8 (0.34) | [−1.47, −0.13]¹ | 0.019² |
| Week 16 | Treatment | −3.9 (0.27)¹ | −3.3 (0.28)¹ | −0.6 (0.38) | [−1.34, 0.17]¹ | 0.129² |

Notes:
LS Mean = least squares mean, SE = standard error.
¹p-values from mixed model repeated measures with treatment, analysis center, visit, and treatment by visit interaction as fixed factors, Baseline Dermatology Life Quality Index Score as a covariate, and subject as a random factor.
²p-values for treatment comparisons using contrasts.

TABLE 7C
Dermatology Life Quality Index, DFD-29 Versus Placebo, MVOR-2

| | | DFD-29 | | Placebo | | | |
|---|---|---|---|---|---|---|---|
| Visit | Parameter | LS Mean (SE) | | LS Mean (SE) | Difference (SE) [95% CB | | p-values |
| Overall | Treatment | −3.5 | (0.28)¹ | −1.7 (0.33)¹ | −1.8 (0.41) | [−2.57, −0.94]¹ | <0.00¹ |
| | Analysis center | | | | | | 0.081¹ |
| | Visit | | | | | | 0.002¹ |
| | Treatment*Visit | | | | | | 0.001¹ |
| | Baseline | | | | | | <0.001¹ |
| Week 2 | Treatment | −2.2 | (0.36)¹ | −1.7 (0.43)¹ | −0.5 (0.55) | [−1.59, 0.57]¹ | 0.353² |
| Week 4 | Treatment | −3.1 | (031)¹ | −1.6 (0.37)¹ | −1.5 (0.47) | [−2.38, 0.53]¹ | 0.002² |
| Week 8 | Treatment | −3.8 | (0.32)¹ | −1.5 (0.39)¹ | −2.3 (0.49) | [3.27, −1.34]¹ | <0.001² |
| Week 12 | Treatment | −4.1 | (0.35)¹ | −2.0 (0.43)¹ | −2.0 (0.54) | [−3.12, −0.97]¹ | <0.001² |
| Week 16 | Treatment | −4.3 | (0.34)¹ | −1.8 (0.43)¹ | −2.5 (0.53) | [−3.51, −1.41]¹ | <0.001² |

Notes:
LS Mean = least squares mean, SE = standard error.
¹p-values from mixed model repeated measures with treatment, analysis center, visit, and treatment by visit interaction as fixed factors, Baseline Dermatology Life Quality Index Score as a covariate, and subject as a random factor.
²p-values for treatment comparisons using contrasts.

TABLE 7D
Dermatology Life Quality Index, DFD-29 Versus Doxycycline, MVOR-2

| | | DED-29 | Doxycycline | | | |
|---|---|---|---|---|---|---|
| Visit | Parameter | LS Mean (SE) | LS Mean (SE) | Difference (SE) [95% CI] | | p-values |
| Overall | Treatment | −3.4 (0.25)¹ | −3.0 (0.25)¹ | −0.5 (0.34) | [−1.14, 0.21]¹ | 0.174¹ |
| | Analysis center | | | | | 0.010¹ |
| | Visit | | | | | <0.001¹ |
| | Treatment*Visit | | | | | 0.402¹ |
| | Baseline | | | | | <0.001¹ |
| Week 2 | Treatment | −2.1 (0.36)¹ | −2.3 (0.36)¹ | 0.1 (0.49) | [−0.83, 1.11]¹ | 0.783² |
| Week 4 | Treatment | −3.0 (0.30)¹ | −2.5 (0.30)¹ | −0.5 (0.41) | [−1.33, 0.30]¹ | 0.217² |
| Week S | Treatment | −3.8 (0.28)¹ | −3.1 (0.27)¹ | −0.7 (0.37) | [−1.41, 0.06]¹ | 0.071² |
| Week 12 | Treatment | −4.0 (0.30)¹ | −3.4 (0.30)¹ | −0.7 (0.42) | [−1.49, 0.15]¹ | 0.110² |
| Week 16 | Treatment | −4.2 (0.32)¹ | −3.6 (0.32)¹ | 0.6 (0.44) | [−1.48, 0.28]¹ | 0.178² |

Notes:
LS Mean = least squares mean, SE = standard error.
¹p-values from mixed model repeated measures with treatment, analysis center, visit, and treatment by visit interaction as fixed factors, Baseline Dermatology Life Quality Index Score as a covariate, and subject as a random factor.
²p-values for treatment comparisons using contrasts.

While the present invention has been illustrated and described with reference to certain exemplary embodiments, those of ordinary skill in the art will understand that various modifications and changes may be made to the described embodiments without departing from the spirit and scope. of the present invention, as defined in the following claims.

1. A method of treating inflammatory lesions in a subject suffering from moderate or severe rosacea, the method comprising:
orally administering a pharmaceutical composition comprising a body weight-independent dose of about 20 mg to about 40 mg of minocycline on a daily basis to the subject,
wherein orally administering the pharmaceutical composition on a daily basis for at least 2 weeks reduces total lesion count in the subject as compared to the total lesion count before administering the pharmaceutical composition, and
wherein the total lesion count comprises a count of papules and pustules on the subject's skin.

2. (canceled)

3. The method of claim 1, wherein the moderate to severe rosacea is characterized by an Investigator's Global Assessment (IGA) score of 3 to 4, respectively, and the pharmaceutical composition reduces the IGA score by at least 2 grades.

4. The method of claim 1, wherein orally administering the pharmaceutical composition reduces the IGA score to 0 or 1.

5. The method of claim 1, wherein the pharmaceutical composition comprises about 30 mg to about 40 mg minocycline.

6. The method of claim 1, wherein the pharmaceutical composition comprises about 40 mg minocycline.

7. The method of claim 1, wherein the pharmaceutical composition comprises minocycline as an immediate release (IR) portion and an extended release (ER) portion in an IR to ER ratio ranging from 1:1 to 1:4.

8. The method of claim 1, wherein the pharmaceutical composition comprises minocycline as an immediate release (IR) portion and an extended release (ER) portion in an IR to ER ratio of 1:3 or 25:75.

9. The method of claim 1, wherein the oral administration on a daily basis for at least 2 weeks comprises 2 weeks, 4 weeks, 8 weeks, 12 weeks, or 16 weeks.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. The method of claim 1, wherein the total lesion count before treatment comprises at least 15 facial papules or pustules.

31. The method of claim 1, wherein after at least 2 weeks, the total lesion count has decreased by at least 30%.

32. The method of claim 1, wherein after at least 2 weeks, the total lesion count has decreased by at least 40%.

33. The method of claim 1, wherein after 16 weeks, the total lesion count has decreased by at least 65%.

34. The method of claim 1, further comprising a reduction in Clinicians Erythema Assessment (CEA) score.

35. The method or the pharmaceutical composition of claim 34, wherein the CEA score is reduced by at least 2 grades.

36. (canceled)

37. The method of claim 1, further comprising a decrease in a Rosacea Quality of Life score.

38. The method of claim 1, further comprising a decrease in a Dermatology Life Quality Index score.

39. The method of claim 1, wherein at 40 mg minocycline on Day 21 compared to Day 1, the interstitial fluid (ISF) AUC₀₋₂₄ and Cₘₐₓ in the subject do not increase by more than 20%.

40. The method of claim 1, wherein at 40 mg minocycline, the interstitial fluid (ISF) AUC₀₋₂₄ and Cₘₐₓ in the subject at Day 1 is at least 80% of the ISF AUC₀₋₂₄ and Cₘₐₓ in the subject at Day 21.

41. The method of claim 1, wherein the plasma AUC₀₋₂₄ and Cₘₐₓ in the subject at Day 1 and Day 21 differ by 15% or less.

42. The method of claim 1, wherein at 20 mg minocycline on Day 21 compared to Day 1, the interstitial fluid (ISF) Cₘₐₓ in the subject increases by about 41% and the plasma Cₘₐₓ increases by about 25%.
